(19) United States
(12) Patent Application Publication
(10) Pub. No.: US 2017/0020939 A1
(43) Pub. Date: Jan. 26, 2017

(54) IMMUNE BALANCE REGULATOR

(71) Applicant: euglena Co., Ltd., Tokyo (JP)

(72) Inventors: Ayaka NAKASHIMA, Kanagawa (JP); Yuta ASAYAMA, Kanagawa (JP); Eriko YOSHIDA, Kanagawa (JP); Osamu IWATA, Kanagawa (JP); Kengo SUZUKI, Kanagawa (JP)

(73) Assignee: EUGLENA CO., LTD., Tokyo (JP)

(21) Appl. No.: 15/302,300

(22) PCT Filed: Apr. 8, 2015
(86) PCT No.: PCT/JP2015/061043

§ 371 Date: Oct. 6, 2016

(30) Foreign Application Priority Data
Apr. 8, 2014 (JP) 2014-079648
Oct. 24, 2014 (JP) 2014-217592
Nov. 7, 2014 (JP) 2014-227332

(51) Int. Cl. A61K 36/02; A23L 33/10; A61K 31/715

(52) U.S. Cl. CPC A61K 36/02; A61K 31/715; A23L 33/10; A23V 2002/00

(57) ABSTRACT

The present invention provides a novel immune balance regulator that regulates an immune balance in a living body. The immune balance regulator contains a Euglena-derived material and regulates an immune balance between Th1, Th2, and Th17, which is a balance between immune responses individually induced by Th1, Th2, and Th17 in a living body. The immune balance regulator regulates the immune balance between Th1, Th2, and Th17 so that immune responses induced by Th1 become relatively dominant over immune responses induced by Th2 or Th17 to improve a physical constitution that has an immune imbalance of Th1, Th2, and Th17 shifted towards Th2 and to prevent or treat a disease associated with the immune imbalance of Th1, Th2, and Th17 shifted towards a Th2 dominance. The immune balance regulator is administered prior to expected onset of a disease associated with the immune imbalance of Th1, Th2, and Th17 shifted towards the Th2 dominance.

### TECHNICAL FIELD

The present invention relates to a novel immune balance regulator.

### BACKGROUND ART

An immune system, which eliminates foreign matter in a living body, has complex mechanisms involving various cells and cytokines.

One of the immune mechanisms is known to control a balance between cellular immunity and humoral immunity. The cellular immunity is a response that eliminates antigens, mainly through the actions of killer T cells and macrophages, while the humoral immunity is a response that eliminates antigens, mainly through the actions of antibodies produced by B cells. The two responses are combined to eliminate antigens.

Accordingly, food and drinks that contain lactic acid bacteria or a processed product therefrom and that balances cellular immunity and humoral immunity to normalize immunity have been proposed (for example, Patent Literature 1).

Although, however, the food and drinks in Patent Literature 1 can regulate an immune balance between cellular immunity and humoral immunity, the food and drinks contain a material derived from lactic acid bacteria and thus have a flavor characteristic of lactic acid bacteria, which limits the application to food and drinks that are appropriate for the flavor characteristic of lactic acid bacteria.

Additionally, it has been found in recent years that helper T cells are classified into three subtypes: type 1 helper T cells (Th1), type 2 helper T cells (Th2), and type 17 helper T cells (Th17), and Th1, which induce cellular immunity, Th2, which induce humoral immunity, and Th17 control disorders associated with various immune responses in mice and humans. It is expected that Th1, Th2, and Th17 are functionally balanced to each other and that maintenance of the balance reduces risk for certain diseases, while loss of the balance leads to onset and progression of various diseases.

It is believed that if a proper Th1/Th2/Th17 balance is lost, and then an imbalance shifted towards a Th1 dominance is created, cellular immunity becomes excessive, which may causes an autoimmune disease such as rheumatoid arthritis. In contrast, it is believed that if the balance is shifted towards a Th2 dominance, humoral immunity becomes excessive, which is likely to cause a disease such as a cancer, immunodeficiency, asthma, dermatitis, an allergic symptom, nephritis, an infection, or a stress disease. In contrast, it is believed that an imbalance shifted towards a Th17 dominance is likely to cause an autoimmune disease such as rheumatoid arthritis.

Thus, it has been found that excess of either cellular immunity, humoral immunity, or Th17 is not desirable and that immune functions properly work when cellular immunity, humoral immunity, and Th17 are well-balanced.

Euglena have been attracting the attention as a biological resource that is promising for use as food, diets, fuels, and the like.

Euglena include as many as 59 nutrients such as vitamins, minerals, amino acids, and unsaturated fatty acids, which represent the majority of the essential nutrients for humans. Studies have demonstrated the feasibility of using Euglena as supplements that provide a balanced combination of various types of nutrients and as food for nutritionally-deprived people in poor regions.

Euglena are in the bottom of the food chain and are eaten by animals. It is more difficult to identify conditions for culturing Euglena, such as light, temperature, and agitation speed, compared with other microorganisms. Thus, it has been considered that Euglena is difficult to culture to a large number. In recent years, however, the inventors of the present invention have developed a technique for culturing Euglena to a large number through their extensive research and have paved the way for a large supply of paramylon.

Euglena are unique living organisms that have animal features such as flagellar motility and also have chloroplast and photosynthesize just like a plant. Although it is expected that Euglena themselves and Euglena-derived materials have many functionalities, many of their functions and mechanisms of generation of the functionalities are still unknown.

Thus, it is desirable to elucidate the functions of Euglena and Euglena-derived materials such as paramylon that now can be supplied in large quantities and the mechanisms of generation of their functionalities and to develop a method for using these materials and the like.

In addition, among the Euglena-derived materials, processed products such as paramylon and amorphous paramylon do not degrade the flavor of food or drinks as a base, when the processed products are incorporated into the food and drinks such as cookies, crackers, chips, Japanese sweets, and smoothie.

### CITATION LIST

### Patent Literature

PATENT LITERATURE 1: Japanese Patent No. 4459938

### SUMMARY OF INVENTION

### Technical Problem

The present invention has been made in view of the foregoing problems, and an object of the present invention is to provide a novel immune balance regulator that regulates the immune balance between Th1, Th2, and Th17 in a living body.

Another object of the present invention is to provide an immune balance regulator that provides a novel method for using Euglena-derived materials.

### Solution to Problem

Through their extensive research on mechanisms for Euglena-derived materials to be involved in immunity, the inventors of the present invention have found that administration of Euglena themselves or Euglena-derived materials such as paramylon or processed products from paramylon to a living body results in promotion of the production of a certain cytokine involved in the immune system and inhibition of the production of other cytokines.

In other words, the inventors of the present invention have found that administration of Euglena or a Euglena-derived material such as paramylon or a processed product from paramylon to a living body in which various cells and cytokines, which have different functions, such as T cells, B cells, and many cytokines, interact with each other to constitute the overall immune system, results in regulation of an immune balance between Th1, Th2, and Th17 in the living body to achieve a proper balance by biasing the balance towards the Th1, Th2, or Th17 dominance, thereby achieving the present invention.

The problem is solved by the immune balance regulator of the present invention, the regulator containing a Euglena-derived material and regulating an immune balance between Th1, Th2, and Th17, which is a balance between immune responses individually induced by Th1, Th2, and Th17 in a living body.

Such regulator can be used as an agent for improving an undesired physical constitution associated with the immune imbalance of Th1, Th2, and Th17 shifted towards a Th1, Th2, or Th17 dominance; an agent for preventing or treating a disease associated with the imbalance shifted towards the Th1, Th2, or Th17 dominance; and the like.

As the regulator can also regulate the immune balance between Th1, Th2, and Th17 in a living body to achieve a proper balance without biasing the balance towards the Th1, Th2, or Th17 dominance, the regulator can be used as an immune balance regulator for controlling the physical condition of healthy individuals who have no disease, the elderly with a weakened immune system, and the like; an agent for improving the physical condition of individuals who have no certain disease but are continuously in a bad physical condition for a certain period of time because the individuals have an immune imbalance shifted towards Th1, Th2, or Th17; and a medicament for enhancing the natural healing power of patients who have a disease associated with the immune imbalance of Th1, Th2, and Th17 shifted towards Th1, Th2, or Th17 in the body to heal themselves.

The immune balance regulator of the present invention uses a Euglena-derived material as the active ingredient. Thus, when the regulator of the present invention is formulated into food, drinks, a supplement, or the like that contain the Euglena-derived material, the active ingredient does not degrade the flavor, which can provide the immune balance regulator in a palatable form.

The immune balance regulator may regulate the immune balance between Th1, Th2, and Th17 so that immune responses induced by Th1 become relatively dominant over immune responses induced by Th2 or Th17. The immune balance regulator may also be used to improve a physical constitution that has an immune imbalance of Th1, Th2, and Th17 shifted towards a Th2 dominance.

Such regulator can improve a bad physical condition that is caused by the immune imbalance of Th1, Th2, and Th17 shifted towards the Th2 dominance and that is not severe enough to need therapy by a doctor and can improve QOL (quality of life).

The physical constitution that has an immune imbalance of Th1, Th2, and Th17 shifted towards the Th2 dominance may be a physical constitution that is susceptible to an infectious or a stress disease.

The immune balance regulator may be used to prevent or treat a disease associated with the immune imbalance of Th1, Th2, and Th17 shifted towards the Th2 dominance.

Such immune balance regulator can be used not only for treatment mainly by a synthetic drug but for promotion of natural healing to restore the immune balance.

Examples of the disease associated with the immune imbalance of Th1, Th2, and Th17 shifted towards the Th2 dominance that can be prevented or treated using the immune balance regulator include cancers, immunodeficiency, asthma, dermatitis, allergic diseases, nephritis, and infections.

The immune balance regulator may be administered prior to expected onset of the disease associated with the immune imbalance of Th1, Th2, and Th17 shifted towards the Th2 dominance.

Such immune balance regulator can prevent expected onset of the disease in advance.

The disease associated with the immune imbalance of Th1, Th2, and Th17 shifted towards the Th2 dominance may be an infectious or a stress disease.

The infection may be influenza, and the immune balance regulator may be used as an anti-influenza agent. The stress disease may be a peptic ulcer, and the immune balance regulator may be used as an agent for preventing or treating the peptic ulcer.

The immune balance regulator may increase the ratio of the IFN-γ production to IL-4 production in the living body. The immune balance regulator may promote the production of IFN-γ and may inhibit the production of IL-4, IL-5, and IL-10 in the living body.

The immune balance regulator may regulate the immune balance between Th1, Th2, and Th17 so that immune responses induced by Th2 become relatively dominant over immune responses induced by Th1 or Th17. The immune balance regulator may be used to improve a physical constitution that has the immune imbalance of Th1, Th2, and Th17 shifted towards a Th1 and/or Th17 dominance. The physical constitution that has the immune imbalance of Th1, Th2, and Th17 shifted towards the Th1 and/or Th17 dominance may be susceptible to diseases such as diabetes, hepatopathy, airway inflammation, host-versus-graft reactions, chronic rheumatoid arthritis, multiple sclerosis, arteriosclerosis, psoriasis, and gastritis. In particular, a physical constitution that has an imbalance shifted towards the Th17 dominance may be susceptible to diseases such as chronic rheumatoid arthritis, multiple sclerosis, psoriasis, and inflammatory bowel disease.

Such immune balance regulator can be used to prevent or treat, for example, a disease associated with the immune imbalance of Th1, Th2, and Th17 shifted towards the Th1 and/or Th17 dominance, such as, for example, diabetes, hepatopathy, airway inflammation, a host-versus-graft reaction, chronic rheumatoid arthritis, multiple sclerosis, arteriosclerosis, psoriasis, and gastritis. In particular, the immune balance regulator can be used to prevent or treat a disease associated with an imbalance shifted towards the Th17 dominance, such as, for example, chronic rheumatoid arthritis, multiple sclerosis, psoriasis, and an inflammatory bowel disease.

The immune balance regulator is used to prevent or treat a disease associated with the immune imbalance of Th1, Th2, and Th17 shifted towards the Th1 and/or Th17 dominance, and the disease may be rheumatoid arthritis. The immune balance regulator may be administered prior to expected onset of the disease associated with the immune imbalance of Th1, Th2, and Th17 shifted towards the Th1 and/or Th17 dominance.

In addition, the Euglena-derived material may be paramylon or a processed product therefrom.

As described above, the immune balance regulator of the present invention uses paramylon or a processed product therefrom as an active ingredient. Thus, when the regulator of the present invention is formulated into food, drinks, a supplement, or the like that contains the paramylon or a processed product therefrom, the active ingredient does not degrade the flavor, which can provide the immune balance regulator in a palatable form.

### Effects of Invention

According to the present invention, the immune balance regulator of the present invention can be used as an agent for improving an undesired physical constitution associated with the immune imbalance of Th1, Th2, and Th17 shifted towards the Th1, Th2, or Th17 dominance; an agent for preventing or treating a disease associated with the imbalance shifted towards the Th1, Th2, or Th17 dominance; and the like to regulate the immune imbalance of Th1, Th2, and Th17 in a living body.

As the regulator can also regulate the immune balance between Th1, Th2, and Th17 in a living body to achieve a proper balance without any imbalance shifted towards the Th1, Th2, or Th17 dominance, the regulator can be used as an immune balance regulator for controlling the physical condition of healthy individuals who have no disease, the elderly with a weakened immune system, and the like; an agent for improving the physical condition of individuals who have no certain disease but are continuously in a bad physical condition for a certain period of time because the individuals have an immune balance shifted towards the Th1, Th2, or Th17 dominance; and a medicament for enhancing the natural healing power of patients who have a disease associated with the immune imbalance of Th1, Th2, and Th17 shifted towards the Th1, Th2, or Th17 dominance in the body to heal themselves.

And the immune balance regulator of the present invention uses paramylon or a processed product therefrom as an active ingredient. Thus, when the regulator of the present invention is formulated into food, drinks, a supplement, or the like that contains paramylon or the processed product therefrom, the active ingredient does not degrade the flavor, which can provide the immune balance regulator in a palatable form.

### BRIEF DESCRIPTION OF DRAWINGS

FIG. 1 is a graph illustrating measurements of IFN-γ in humans who took the immune balance regulator of Example 2 of the present invention for 8 weeks.

FIG. 2 is a graph illustrating measurements of IL-4 in humans who took the immune balance regulator of Example 2 of the present invention for 8 weeks.

FIG. 3 is a graph illustrating calculations of IFN-γ/IL-4 in humans who took the immune balance regulator of Example 2 of the present invention for 8 weeks.

FIG. 4 is a graph illustrating measurements of IL-6 in humans who took the immune balance regulator of Example 2 of the present invention for 8 weeks.

FIG. 5 is a graph illustrating measurements of IL-12p70 in humans who took the immune balance regulator of Example 2 of the present invention for 8 weeks.

FIG. 6 is a graph illustrating measurements of IL-10 in humans who took the immune balance regulator of Example 2 of the present invention for 8 weeks.

FIG. 7 is a graph illustrating measurements of IL-5 in humans who took the immune balance regulator of Example 2 of the present invention for 8 weeks.

FIG. 8 is a graph illustrating measurements of monocytes in humans who took the immune balance regulator of Example 2 of the present invention for 8 weeks.

FIG. 9 is a graph illustrating survival rates of mice that took paramylon prepared in Example 2 of the present invention, amorphous paramylon of Example 3, or Euglena of Example 1 by a diet admixture for a week and then were infected with influenza virus.

FIG. 10 is a graph illustrating survival rates of mice that took the paramylon prepared in Example 2 of the present invention, the amorphous paramylon of Example 3, or the Euglena of Example 1 by a diet admixture for 2 weeks and then were infected with influenza virus.

FIG. 11 is a graph illustrating measurements of virus titers in mice at day 2 after infection of the mice with influenza virus after the mice took the paramylon prepared in Example 2 of the present invention, the amorphous paramylon of Example 3, or the Euglena of Example 1 by a diet admixture for a weeks.

FIG. 12 is a graph illustrating measurements of virus titers in mice at day 2 after infection of the mice with influenza virus after the mice took the paramylon prepared in Example 2 of the present invention, the amorphous paramylon of Example 3, or the Euglena of Example 1 by a diet admixture for 2 weeks.

FIG. 13 is a graph illustrating measurements of virus titers in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 14 is a graph illustrating measurements of IL-113 in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 15 is a graph illustrating measurements of IL-6 in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 16 is a graph illustrating measurements of IL-10 in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 17 is a graph illustrating measurements of IL-12 (p70) in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 18 is a graph illustrating measurements of IFN-γ in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 19 is a graph illustrating measurements of TNF-α in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 20 is a graph illustrating measurements of IFN-β in mice at days 1, 2, and 3 after infection of the mice with influenza virus after the mice took the paramylon prepared in Example 2 of the present invention, the amorphous paramylon of Example 3, or the Euglena of Example 1 by a diet admixture for 2 weeks.

FIG. 21 is a graph illustrating the amounts of diets for the respective groups taken by rats for 14 days in Study Example 6.

FIG. 22 is a graph illustrating the body weights of rats that took the diets for the respective groups for 14 days in Study Example 6.

FIG. 23 are photographs illustrating gastric ulcers in representatives in the respective groups in Study Example 6.

FIG. 24 is a graph illustrating measurements of the areas of gastric ulcers in the respective groups in Study Example 6.

FIG. 25 are photographs illustrating iNOS mRNA bands, COX-2 mRNA bands, and β-actin mRNA bands detected in Study Example 6.

FIG. 26 is a graph illustrating iNOS/β-actin in Study Example 6.

FIG. 27 is a graph illustrating COX-2/β-actin in Study Example 6.

FIG. 28 is a graph illustrating arthritis scores of mouse models of collagen arthritis in Study Example 7.

FIG. 29 is a graph illustrating measurements of anti-collagen IgG in mouse models of collagen arthritis in Study Example 7.

FIG. 30 is a graph illustrating measurements of cytokines (IL-17) in mouse models of collagen arthritis in Study Example 7.

FIG. 31 is a graph illustrating measurements of cytokines (IFN-γ) in mouse models of collagen arthritis in Study Example 7.

FIG. 32 illustrates a method for preparing a pathological sample of a knee joint to evaluate the tissue of the knee joint in mouse models of collagen arthritis in Study Example 7.

FIG. 33 are photographs of the pathological sample of the tissue of the left knee joint of a mouse model of collagen arthritis of an untreated group in Study Example 7.

FIG. 34 are photographs of the pathological sample of the tissue of the left knee joint of a mouse model of collagen arthritis of a control group in Study Example 7.

FIG. 35 are photographs of the pathological sample of the tissue of the left knee joint of a mouse model of collagen arthritis of a Euglena group in Study Example 7.

FIG. 36 are photographs of the pathological sample of the tissue of the left knee joint of a mouse model of collagen arthritis of a paramylon group in Study Example 7.

FIG. 37 are photographs of the pathological sample of the tissue of the left knee joint of a mouse model of collagen arthritis of an amorphous paramylon group in Study Example 7.

FIG. 38 are photographs of the pathological sample of the tissue of the left knee joint of a mouse model of collagen arthritis of an emulsion paramylon group in Study Example 7.

FIG. 39 is a graph illustrating the ratio of IL-17 cells production in CD4 positive T cells in mouse models of collagen arthritis in Study Example 7.

### DESCRIPTION OF EMBODIMENTS

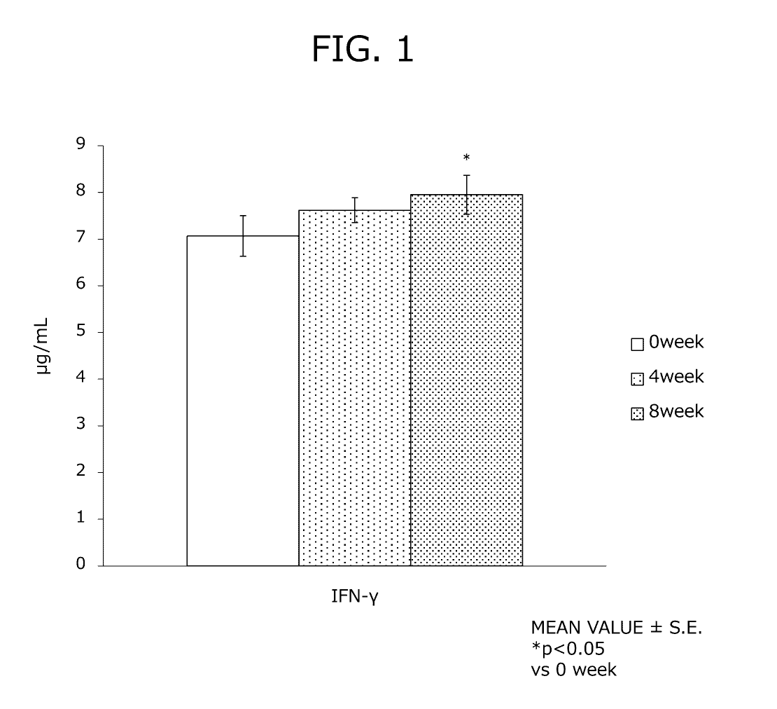

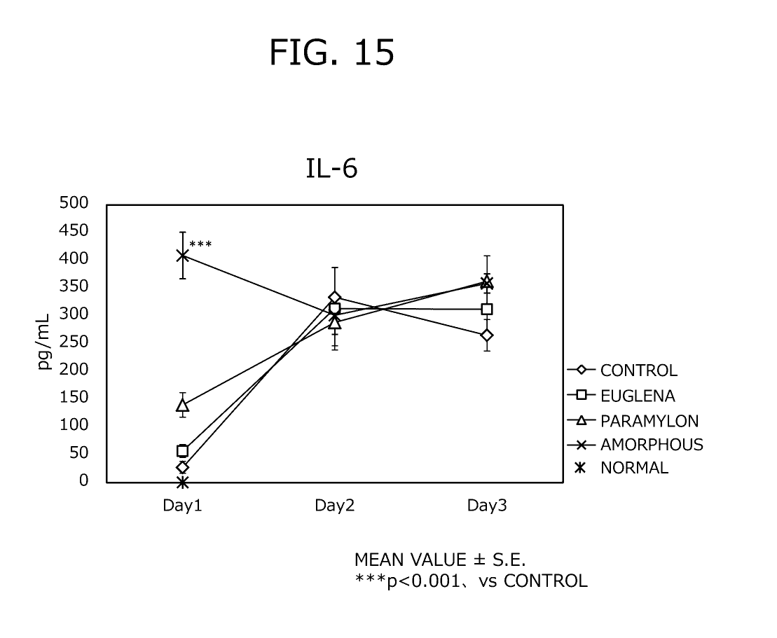

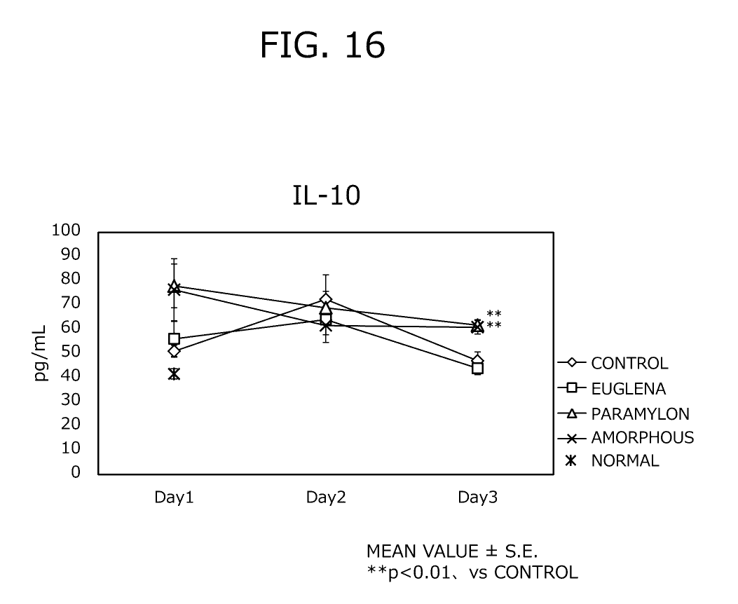

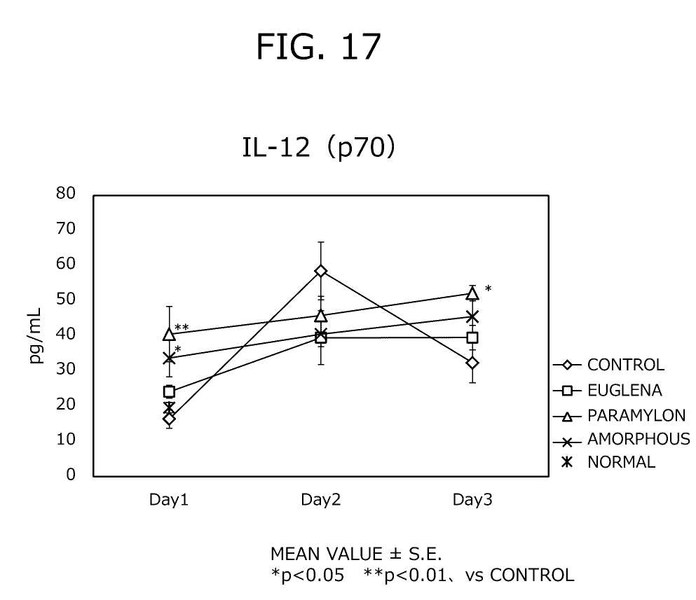

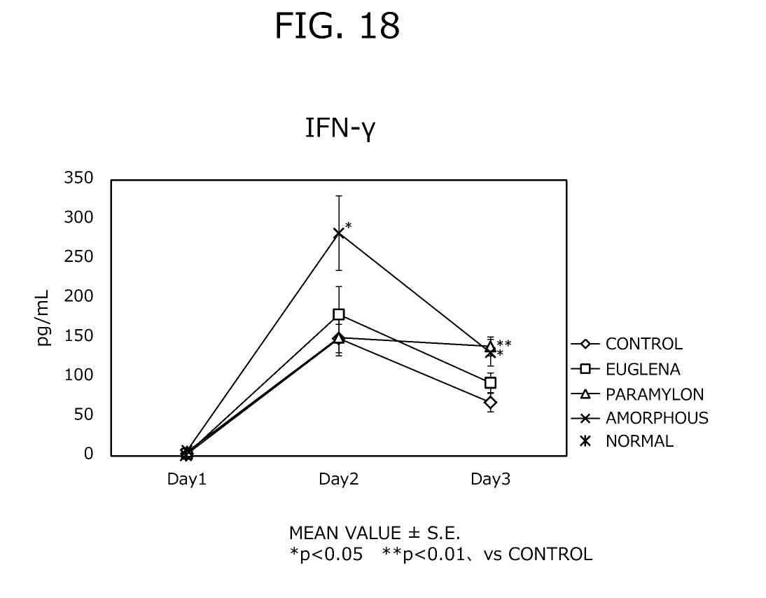

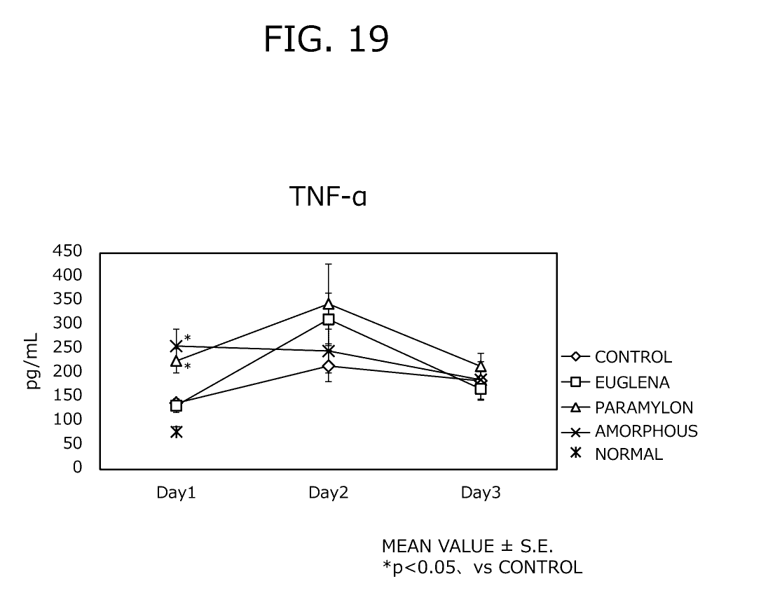

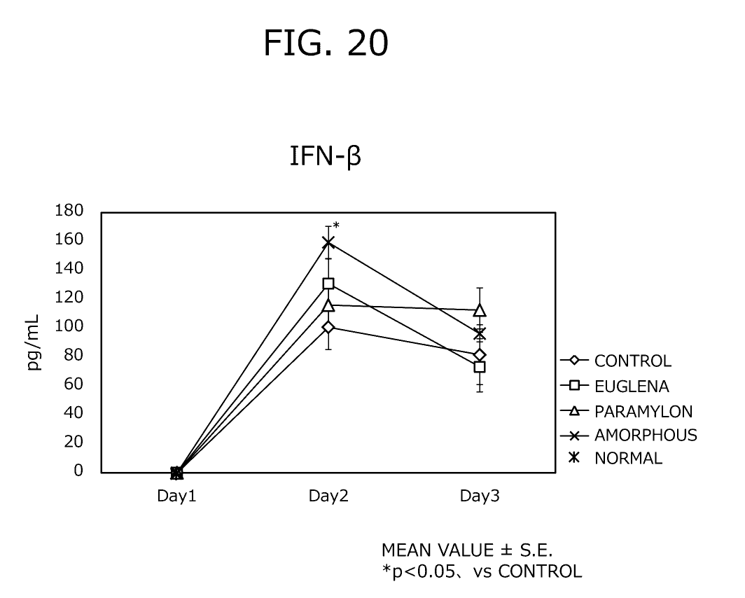

Now, an immune balance regulator according to an embodiment of the present invention will be described with reference to FIGS. 1-39.

### <<Mechanism of Cellular Immunity and Humoral Immunity>>

Helper T cells (naive T cells) migrate through the lymphoid tissue in a body and are repetitively contacted with antigen-presenting cells until the T cells encounter their specific antigen such as a virus or a microbe. Naive T cells that have encountered their specific antigen repetitively proliferate and differentiate into immature effector T cells (Th0), which subsequently differentiate into Th1, Th2, or Th17 effector T cells by stimulation with cytokines and co-stimulation with antigen-presenting cells. The effector T cells are categorized into type 1 helper T cells (Th1), type 2 helper T cells (Th2), and type 17 helper T cells (Th17) depending on their produced cytokines.

Th1, Th2, and Th17 produce different types of cytokines and activate separate immune systems and more particularly, respectively activate cellular, humoral, Th17-specific immune systems.

In the cellular immunity, killer T cells themselves are responsible for the immune responses. The T cells cluster around antigens, which are subsequently attacked and destroyed by the T cells.

In the humoral immunity, antibody-producing cells produce antibodies, which are responsible for the immune responses. The antibodies are present in blood, and antigen-antibody reactions occur throughout the body. Different antibodies are produced against different antigens and specifically bind to the antigens to inhibit the action of the antigens.

In other words, in the cellular immunity, T cells directly attack antigens, while in the humoral immunity, antibodies are produced, and the antibodies specifically bind to antigens to inactivate the antigens.

The process of the humoral immunity is as follows: When antigens such as pathogens enter a body, the antigens are processed by phagocytosis by macrophages. The macrophages become antigen-presenting cells, which then display information on the endocytosed antigen to helper T cells. The helper T cells recognize the antigen information and then release cytokines to stimulate specific B cells to proliferate. The proliferated B cells become antibody-producing cells to produce antibodies. The helper T cells also have a role in assisting in the production of the antibodies. Antigen-antibody reactions occur between the antigens and the antibodies secreted into bodily fluids, and then the antigens are removed by agglutination, precipitation, and lysis.

In the cellular immunity, macrophages process antigens that have entered a body and present information on the antigens to helper T cells. The helper T cells that have received the information stimulate killer T cells. The stimulated killer T cells proliferate and directly react with the antigens to inactivate the antigens.

Th1 produce IFN-γ and IL-2 cytokines, which activate cellular immunity, and increase the activities of killer T cells and macrophages. IL-2 induce the proliferation of B cells and proliferation and the activation of Th1. IFN-γ also activate macrophages. TNF-β induce the production of IFN-γ and activate macrophages, thereby participating in the cellular immunity.

The differentiation into Th1 requires IL-12 secreted by antigen-presenting cells, and IFN-γ, which are produced by Th1 and the like, promote the differentiation of Th0 into Th1. IL-10 produced by Th2 inhibit the production of IL-12 by macrophages and thus inhibit the production of IFN-γ by Th1, thereby indirectly inhibiting the differentiation of Th0 into Th1.

Th2 produce IL-4, IL-5, and IL-10 cytokines, which activate humoral immunity, and promote the activation of B cells and the production of antibodies. It is believed that PGE₂ that are secreted by macrophages during antigen presentation play an important role in the differentiation into Th2. IL-4 and IL-6 produced by Th2 promote the differentiation of Th0 into Th2. IFN-γ produced by Th1 inhibit the differentiation of Th0 into Th2. IL-4 also activates and induce the proliferation of B cells; inhibit the activation of Th1 and macrophages; and induce the proliferation of Th2. IL-5 induces the proliferation and the differentiation of B cells. PGE₂ promote the differentiation of Th0 into Th2 and inhibits the production of IFN-γ.

Th17 are a new T cell subset that has been discovered in recent years and is said to be involved in the onset of autoimmune diseases. The differentiation of Th0 into Th17 is induced by stimulation with TGF-β and IL-6. Th17 cells themselves produce IL-17 through expression of IL-23. Th17 also produce IL-2, IL-6, TNF-α, and the like.

### <<Immune Balance Regulator>>

In this specification, the immune balance between Th1, Th2, and Th17 refers to a balance between immune responses individually induced by Th1, Th2, and Th17. When the immune responses individually induced by Th1, Th2, and Th17 are balanced to each other, there is no imbalance.

In contrast, when immune responses induced by any of effector T cells including Th1, Th2, and Th17 are excessive compared with other immune responses, there is a bias towards the excessive effector T cells.

In this specification, examples of “regulation of the immune balance so that immune responses induced by Th1 become relatively dominant over immune responses induced by Th2 or Th17” include change of a condition in which immune responses induced by Th2 or Th17 are excessive compared with immune responses induced by Th1 to a condition in which the immune responses by Th2 or Th17 are not excessive by inhibiting the immune responses by Th2 or Th17, or inducing the immune responses by Th1.

Examples of “regulation of the immune balance so that immune responses induced by Th2 become relatively dominant over immune responses induced by Th1 or Th17” include change of a condition in which immune responses induced by Th1 or Th17 are excessive compared with immune responses induced by Th2 to a condition in which the immune responses by Th1 or Th17 are not excessive by inhibiting the immune responses by Th1 or Th17 or inducing the immune responses by Th2.

The phrase “become relatively dominant over” includes a case in which immune responses by an effector T cell are not dominant over immune responses by another effector T cell, but are more greatly induced, accelerated, or activated compared with the immune response before the regulation, as well as a condition in which immune responses by an effector T cell are greater than immune responses by another effector cells.

The phrase “become relatively dominant over” also includes a case in which responses by an effector T cell become relatively greater than responses by another effector T cell as a result of inhibition of the immune responses by another effector T cell. In this case, it is only necessary that the responses by an effector T cell become relatively dominant over the responses by another effector T cell, irrespective of the fact whether the responses by the former effector T cell is accelerated or inhibited.

The immune balance between Th1, Th2, and Th17 is also regulated by inhibiting or promoting the differentiation of naive T cells into Th1, Th2 or Th17.

The immune balance regulator of the embodiment contains a Euglena-derived material and regulates the immune balance between Th1, Th2, and Th17 in a living body, i.e., the balance between cellular immunity, humoral immunity, and immunity by Th17.

The immune balance regulator of the embodiment regulates the balance between Th1, Th2, and Th17 by shifting the balance towards cellular immunity induced by Th1 and/or humoral immunity induced by Th17 or Th2 and preferably maintains the proper balance by shifting a biased balance to eliminate a bias towards any of Th1, Th2, or Th17.

Preferably, the Euglena-derived material contains paramylon, and the examples include Euglena, dried Euglena products, paramylon, paramylon powder, and processed products from paramylon. The Euglena-derived material may also be Euglena or dried Euglena to which paramylon or a processed product from paramylon has been added.

As Euglena cells, Euglena gracilis (E. gracilis) and, in particular, the strain Euglena gracilis (E. gracilis) Z can be used. The Euglena may also be species such as Euglena gracilis Klebs, Euglena gracilis var. bacillaris, the strain SM-ZK (chloroplast deficient strain), which is a mutant derived from the strain Euglena gracilis (E. gracilis) Z, var. bacillaris, or β-1,3-glucanase derived from a genetic mutant strain such as chloroplast mutants thereof, Euglena intermedia, Euglena piride, and other Euglena such as, for example, Astaia longa.

Euglena generally live in fresh water such as pools and ponds, and thus Euglena may be isolated from such water. Alternatively, any previously-isolated Euglena may be used.

The Euglena in the present invention encompass all mutant strains. The mutant strains encompass those produced through genetic techniques such as, for example, recombination, transduction, and transformation.

Culture for cultivating Euglena cells may be, for example, culture supplemented with a nutrient salt such as a nitrogen source, a phosphorus source, or a mineral. For example, modified Cramer-Myers medium (1.0 g/L of (NH₄)₂HPO₄, 1.0 g/L of KH₂PO₄, 0.2 g/L of MgSO₄.7H₂O, 0.02 g/L of CaCl₂.2H₂O, 3 mg/L of Fe₂(SO₂)₃.7H₂O, 1.8 mg/L of MnCl₂.4H₂O, 1.5 mg/L of CoSO₄.7H₂O, 0.4 mg/L of ZnSO₄.7H₂O, 0.2 mg/L of Na₂MoO₄.2H₂O, 0.02 g/L of CuSO₄.5H₂O, 0.1 mg/L of thiamine hydrochloride (vitamin B₁), cyanocobalamin (vitamin B₁₂), (pH 3.5)) may be used. The (NH₄)₂HPO₄ may be displaced by (NH₄)₂SO₄ or NH₃aq. Known Hutner medium or Koren-Hutner medium prepared according to the description in “Euglena—Physiology and Biochemistry” (Kitaoka, S (ed.), Gakkai Shuppan Center, K.K.) may also be used.

The culture preferably has a pH of 2 or higher, and preferably 6 or less and more preferably 4.5 or less. In the culture having an acidic pH, photosynthetic microorganisms can grow better than other microorganisms, which can prevent contamination.

Euglena cells may be cultivated using an open pond process that directly uses sun light, or a light collection process that collects sunlight by light collector, which is subsequently transmitted through fiber optics or the like to a fermenter tank where the cells are exposed to the light for photosynthesis.

Euglena cells may also be cultivated using, for example, a fed-batch process. Euglena cells may be cultivated using any liquid cultivation process such as flask cultivation, fermenter cultivation, batch cultivation, semi-batch cultivation (fed-batch cultivation), or continuous cultivation (perfusion cultivation).

Euglena cells can be cultivated using a known fermenter such as an open pond fermenter, a raceway fermenter, or a tubular fermenter or a laboratory fermenter such as a Sakaguchi flask, an Erlenmeyer flask, or a reagent bottle. Euglena utilize CO₂, and thus if Euglena is cultivated using the Cramer-Myers medium, which is an autotrophic medium, the medium is preferably gassed with air containing 1-5% CO₂. It is also preferred to add about 1-5 g of ammonium phosphate per liter of the medium to sufficiently develop chloroplasts. Suitably, the cultivation is carried out generally at a temperature of from 20-34° C. and particularly from 28-30° C. Euglena usually enter the logarithmic growth phase at 2-3 days after the initiation of the cultivation and reach the stationary phase at 4-5 days, although these depend on the cultivation conditions.

Euglena may be cultivated under light exposure (light cultivation) or without light exposure (dark cultivation).

Euglena cells may be isolated by, for example, centrifugation of the culture or simple sedimentation.

Paramylon is a polymer of about 700 glucose units polymerized through β-1,3-linkage (β-1,3-glucan) and a reserve polysaccharide contained in Euglena. Paramylon particles have a flattened spheroid shape and are formed of helical β-1,3-glucan chains.

Paramylon exists, as granules, in all species and varieties of Euglena cells, and the number, shape, and particle uniformity vary with the species.

Paramylon is composed of glucose only, and paramylon from wild type E. gracilis Z and chloroplast deficient mutant SM-ZK has an average degree of polymerization of about 700 glucose units.

Although Paramylon is insoluble in water and hot water, Paramylon is soluble in dilute alkali, concentrated acid, dimethyl sulfoxide, formaldehyde, and formic acid.

Paramylon in E. gracilis Z and paramylon in E. gracilis var. bacillaris SM-L1 respectively have an average density of 1.53 and 1.63.

X-ray analysis using a powder pattern technique shows that paramylon assumes a gentle helical configuration of three linear β-glucan chains wound together in a right-hand direction. Some of the glucan molecules aggregate to form paramylon granules. The paramylon granules have a large number of crystal structures, which make up about 90%, and a compound having the highest crystal structure ratio among polysaccharides. Paramylon is also less likely to contain water (“Euglena—Physiology and Biochemistry” (Kitaoka, S (ed.), Gakkai Shuppan Center, K.K.)).

Paramylon (from Euglena Co., Ltd.) has a median size as a particle size distribution of 1.5-2.5 μm as measured on a laser diffraction/scattering particle size distribution analyzer.

Paramylon particles are isolated from cultivated Euglena by any suitable technique and are processed into fine particles, which are usually provided as powder.

For example, paramylon particles can be obtained by (1) cultivation of Euglena cells in any suitable medium, (2) separation of the Euglena cells from the medium, (3) isolation of paramylon from the separated Euglena cells, (4) purification of the isolated paramylon, and optionally, (5) cooling and subsequent lyophilization.

Paramylon can be isolated using, for example, a nonionic or anionic surfactant that is largely biodegradable. In practice, paramylon can be purified simultaneously with the isolation step.

Isolation of paramylon from Euglena and purification are well known and described in, for example, E. Ziegler, “Die naturlichen und kunstlichen Aromen” Heidelberg, Germany, 1982, Chapter 4.3 “Gefriertrocken”, DE 43 28 329, and Japanese Patent Application National Publication No. 2003-529538.

Examples of the processed product from paramylon include amorphous paramylon and emulsion paramylon.

Amorphous paramylon is produced by amorphization of crystalline paramylon derived from Euglena.

Amorphous paramylon used in the embodiment has a relative crystallinity of 1-20%, as compared with that of crystalline paramylon produced from Euglena by a known method.

The relative crystallinity is determined by a method described in Japanese Patent No. 5612875.

In particular, amorphous paramylon and paramylon are individually ground in a pulverizer (MM400 ball mill from Retsch) at 20 oscillations per second for 5 minutes, and then scanned with an X-ray diffractometer (H'PertPRO from Spectris Co., Ltd.) at a tube voltage of 45 KV, a tube current of 40 mA, and 2θ in the range of from 5° to 30° to obtain diffraction peaks Pc for paramylon and Pa for amorphous paramylon at 2θ of about 20°.

The Pc and Pa values are used to calculate the relative crystallinity of amorphous paramylon as follows:

Relative Crystallinity of Amorphous Paramylon=Pa/Pc×100(%)

Amorphous paramylon used in the embodiment is prepared by treating crystalline paramylon powder with alkali, neutralizing the treated product with acid, washing the product, removing the water, and then drying the product according to a method described in Japanese Patent No. 5612875.

Processed products from paramylon include water-soluble paramylon, sulfated paramylon, and the like that are obtained by chemically or physically treating paramylon by various other known methods, and paramylon derivatives.

The emulsion paramylon is named after its production method and physical properties that are similar to those of emulsion. The emulsion paramylon is obtained by adding water to paramylon to produce fluid and ejecting the fluid from a narrow bore nozzle at a very high pressure to cause the fluid to collide with a collision surface. The emulsion paramylon is a processed paramylon swelled by binding to over 4 times as much water as paramylon.

The emulsion paramylon can be obtained by adding a water-soluble solvent to solid such as powder to form a slurry, ejecting the slurry from a narrow bore nozzle at a very high pressure to cause the slurry to collide with a collision surface one or more times using a known device for improving a physical property (for example, a device as described in Japanese Unexamined Patent Application Publication No. 2011-88108 and Japanese Unexamined Patent Application Publication No. H06-47264) at a nozzle-outlet pressure of 245 MPa.

The emulsion paramylon has a median size as a particle size of 7 μm or more, which is 5 times or more of the size of paramylon, as measured on a laser diffraction/scattering particle size distribution analyzer. Observation under a photoelectron microscope indicates that the particles are in contact with adjacent particles and are swelled by binding to over 4 times as much water as paramylon.

While the slurry produced by combining a paramylon raw material and water is flowable fluid, the emulsion paramylon, which is a dispersion of paramylon in water, has increased viscosity, which causes the emulsion paramylon to stick to hands, and elasticity, which provide a glue-like touch.

Although in this specification, the resultant processed paramylon is named emulsion paramylon after its processing method and physical properties, it is not clear whether the processed paramylon is emulsified. The paramylon is just swelled by binding to water.

The immune balance regulator of the embodiment can be used in a composition such as a pharmaceutical composition, a food composition, or a cosmetic composition that contains the immune balance regulator.

The immune balance regulator of the embodiment can be used to improve a physical constitution that has the immune imbalance of Th1, Th2, and Th17 shifted towards the Th2 dominance and to prevent or treat a disease associated with the immune imbalance of Th1, Th2, and Th17 shifted towards the Th2 dominance. With regard to the disease associated with the immune imbalance of Th1, Th2, and Th17 shifted towards the Th2 dominance, the immune balance regulator can be used to prevent or treat, for example, a cancers, immunodeficiency, asthma, dermatitis, an allergic disease, nephritis, an infection, or the like, and to finely improve the physical condition after the treatment as an adjunct.

An Allergic disease occurs due to an excessive immune response to a specific antigen. The immune balance regulator of the embodiment is used against various allergic diseases such as atopic dermatitis, pollinosis, allergic rhinitis, allergic conjunctivitis, and type I-IV allergies.

The immune balance regulator of the embodiment is used to prevent or treat an infection and finely improve the physical condition after treatment as an adjunct by regulating the immune balance. The immune balance regulator can be used as, for example, an anti-viral agent such as an anti-influenza agent for preventing infection of or treating a viral disease such as influenza. The immune balance regulator of the embodiment has the effect of inhibiting infection and onset upon exposure to a virus such as an influenza virus. The immune balance regulator of the embodiment is used against type B and C influenza viruses as well as type A influenza viruses such as H1N1, H2N2, and H3N2.

Some of anti-viral agents and anti-influenza agents that contain a Euglena-derived material have not been conventionally known.

Some infections such as influenza can become severe. Such infections are prevented by a vaccine or treated by administration of a therapeutic agent depending on the infections, but some vaccines and therapeutic agents have a side effect. There has been a need for an agent for treating and preventing infections without any side effects.

The immune balance regulator of the embodiment can be administered as food, and use of a material derived from Euglena, which can be produced in large quantities, as an anti-viral agent or an anti-influenza agent can provide an anti-viral agent or an anti-influenza agent that can be easily produced, processed, handled, and taken.

Moreover, the immune balance regulator can be administered as an anti-influenza agent in the form of food, and use of a Euglena-derived material having no side effects can provide an anti-influenza agent that can be administered for a long period. Thus, the agent can be administered throughout the year, which can improve the immune system itself in a living body and simultaneously provide a great anti-influenza effect constantly.

It has been found in recent years that some immune responses are promoted by stress. Glucocorticoid and catecholamine, which are increased under stress, inhibit the production of IL-12 in antigen-presenting cells and enhance the production of IL-10. Stimulation with stress strongly inhibits Th1 responses, which induce cellular immunity, thereby driving immune responses towards Th2.

Thus, the immune balance regulator of the embodiment can be used to prevent or treat a stress disease including, for example, peptic ulcers such as gastric ulcers and duodenal ulcers and to finely improve the physical condition after treatment as an adjunct.

Peptic ulcers refer to a condition in which partially lost epithelia are deep within the mucosal lining of the gastrointestinal tract. Causes of ulcers are generally considered to be lost balance between functions of aggressive factors such as gastric acid, pepsin, stress, Helicobacter pylori (hereinafter referred to as “H. pylori”) and nonsteroidal anti-inflammatory drugs (hereinafter referred to as “NSAID”) and protective factors for gastrointestinal mucosa, i.e., mucus and mucosal barriers, blood flow and microcirculation, growth factors, and prostaglandin.

Gastric ulcers are caused mainly by weakened defense mechanisms for gastric mucosa. Infection with H. pylori, NSAID, and stress weaken the defense mechanisms to cause damage to gastric mucosa, which causes an ulcer. Duodenal ulcers are caused by increased secretion of gastric acid, which causes damage to duodenal mucosa, which is vulnerable to attack by gastric acid. Infection with H. pylori also weakens duodenal mucosa. High-fat meals and the like cause increased secretion of gastric acid.

The three most common causes of gastric and duodenal ulcers are infection with H. pylori, nonsteroidal anti-inflammatory drugs (NSAID), and stress.

The immune balance regulator of the embodiment can be administered to those at high risk for developing a peptic ulcer such as, for example, those having mental stress, those after completion of treatment of their peptic ulcer, those after completion of removal of H. pylori, and those after failure to remove H. pylori.

The immune balance regulator of the embodiment can be continuously administered for a long period to those in an environment where they are susceptible to psychological and social stress such as, for example, those who work or live in an environment where they are susceptible to mental stress and those who is preparing for a test or the like.

To those with a body weight of from 40-90 kg, the immune balance regulator of the embodiment is administered in an amount so that paramylon or a processed product from paramylon is given at a dose of 0.05 g or more and preferably 1 g or more per day.

As the immune balance regulator of the embodiment exhibits a greater effect of driving Th1/Th2 immune balance back towards Th1 at week 8 after administration than at week 4, administration of the regulator for a longer period can provide a greater effect of regulating the immune balance.

If it is known that an allergic individual is exposed to a specific antigen in a certain season, the individual is expected to develop a disease in the season. Thus, continuous administration of the immune balance regulator of the embodiment is started, for example, 1 week or earlier and preferably a year or earlier before the beginning of the season improves the immune balance in the individual and improves the basic immunity, thereby improving the allergic constitution and inhibiting allergic symptoms when the individual is exposed to some amount of antigens.

There are cases in which unexplained unpleasant symptoms such as cough, a runny nose, sneezing, and headache are associated with an allergic constitution and are caused by the immune imbalance of Th1, Th2, and Th17 towards the Th2 dominance. In such case, the immune balance regulator of the embodiment can be used as an agent for alleviating the unexplained unpleasant symptoms.

The immune balance regulator of the embodiment inhibits the onset of an infection by regulating the immune balance. The immune balance regulator of the embodiment may also be continuously administered as an anti-influenza agent prior to the initiation of the influenza season to alleviate the symptoms when the infection is developed. For example, continuous administration for 1 week to 1 year prior to the influenza season regulates the immune balance to improve the immunity, thereby making the recipients less prone to the infection even if the recipients are exposed to some amount of the virus.

The immune balance regulator of the embodiment may be continuously administered as an agent for preventing a peptic ulcer to those who are in an environment where they are susceptible to mental stress, such as an environment in which a natural disaster, large-scale fires, an accident, a crime, a war has occurred, those who are receiving a nonsteroidal anti-inflammatory drug (NSAID), and the like. The continuous administration to those who are in an environment where they are prone to develop a peptic ulcer regulates the immune balance to improve the immunity, thereby making them less prone to develop a peptic ulcer.

Continuous administration of the immune balance regulator of the embodiment throughout the year regulates the immune balance to improve the immunity throughout the year and to improve the physical constitution, thereby making the recipients less prone to develop influenza and other common infections even if the recipients are little tired.

The immune balance regulator of the embodiment can also be used to improve a physical constitution that has an immune imbalance of Th1, Th2, and Th17 shifted towards Th1 and/or Th17 and to prevent or treat a disease associated with the immune imbalance of Th1, Th2, and Th17 shifted towards Th1 and/or Th17. With regard to the disease associated with the immune imbalance of Th1, Th2, and Th17 shifted towards the Th1 and/or Th17 dominance, the immune balance regulator can be used to prevent or treat, for example, diabetes, hepatopathy, airway inflammation, a host-versus-graft reaction, chronic rheumatoid arthritis, multiple sclerosis, arteriosclerosis, psoriasis, gastritis, or the like and to finely improve the physical condition after the treatment as an adjunct. Especially, the immune balance regulator can be used to prevent or treat a disease associated with the imbalance shifted towards Th17, such as, for example, chronic rheumatoid arthritis, multiple sclerosis, psoriasis, or inflammatory bowel disease and to finely improve the physical condition after the treatment as an adjunct.

Rheumatoid arthritis is a disorder that causes joints swelling and pain in the hands and feet as the autoimmunity affects the joints in the hands and feet. As the disorder progresses, bone and cartilage destruction causes joint stiffness, thereby severely affecting the daily lives.

No existing pharmaceutical agents against rheumatoid arthritis can provide all of the following effects: remission of rheumatic inflammation, inhibition of progress of joint destruction, and quick effects. And some of the agents have a strong side effect, while others have a weak side effect, and thus these agents are complementarily combined in the pharmacotherapy. However, conventional antirheumatic agents generally exhibit large variation in effects with individuals. An agent may be effective for some cases, while the agent may not be effective for other cases.

Conventional antirheumatic agents also have a high incidence of a side effect and a slow onset of effect in the range of from about 2 weeks to 3 months. The antirheumatic agents cannot be administered until rheumatoid arthritis is diagnosed definitely, due to its high incidence of a side effect. A research on the actual situation (Pfizer Japan Inc., “Research on Actual Situation of 500 Patients with Rheumatoid Arthritis”, Nov. 24, 2011) reports that it took 3 or more months to make definitive diagnosis in over 50% or more of patients with rheumatoid arthritis, and it took 6 months or more from recognition of a symptom to initiation of administration of an antirheumatic agent in over 80% of the patients. It is not uncommon that it took 6-12 months or more from recognition of a symptom to onset of effect of the antirheumatic agent. In addition, the antirheumatic agents are initially administered at a low dose to determine the effects and the presence of a side effect, which is a factor that delays onset of the effects of the antirheumatic agents.

Accordingly, there has been a need for an antirheumatic agent that has little side effect, that exhibits small variation in effects with cases, and that can be used for a living individual who are not diagnosed definitively as rheumatoid arthritis.

Use of a material derived from Euglena, which can be eaten as food and can be produced in large quantities, as an antirheumatic agent and a prophylactic agent against rheumatism can provide an antirheumatic agent and a prophylactic agent against rheumatism that can be easily produced, processed, handled, and taken.

Thus, the immune balance regulator of the embodiment can be administered as an inhibitor of rheumatoid arthritis, a prophylactic agent against rheumatoid arthritis, and a therapeutic agent for rheumatoid arthritis that have no side effects and that can treat immune abnormality associated with rheumatoid arthritis. The immune balance regulator can also be administered as an inhibitor of rheumatoid arthritis, a prophylactic agent against rheumatoid arthritis, and a therapeutic agent for rheumatoid arthritis that can be administered prior to onset or diagnosis of rheumatoid arthritis.

### <<Pharmaceutical Composition>>

In the medical industry, a pharmaceutical composition that has the effect of regulating the immune balance can be provided by formulating a Euglena-derived material in an amount sufficient to effectively provide the effect of regulating the immune balance, a pharmaceutically acceptable carrier, and a pharmaceutically acceptable additive. The pharmaceutical composition may be a pharmaceutical drug or a quasi-drug.

The pharmaceutical composition may be used internally or externally. In particular, the pharmaceutical composition may be used in a dosage form such as an oral agent; an injectable such as an intravenous injectable, a subcutaneous injectable, an intradermal injectable, an intramuscular injectable, and/or an intraperitoneal injectable; a transmucosal agent; or a transdermal agent.

The dosage form of the pharmaceutical composition can be appropriately determined depending on the administration mode, and the example include solid formulations such as tablets, granules, capsules, and powders; and liquid formulations such as solutions and suspensions; and semi-solid formulations such as ointments and gels.

### <<Food Composition>>

In the food industry, a food composition that has the effect of regulating the immune balance can be provided by formulating, into various food a Euglena-derived material as a food material in an amount sufficient to effectively provide the effect of regulating the immune balance in a living body. Thus, the present invention can provide a food composition that is provided with an indication that the composition is for regulation of the immune balance in the food industry. Examples of the food composition include general food, food for specified health use, nutritional supplement food, functional food, food for inpatients, and supplements. The composition may also be used as a food additive.

Examples of the food composition can include seasoning, processed meat products, processed crop products, drinks (such as soft drinks, alcoholic drinks, carbonated drinks, milk drinks, fruit drinks, teas, coffee, and nourishing drinks), powdered drinks (such as powdered juice and powdered soup), concentrated drinks, confectionery (such as candies, cookies, crackers, gums, gummi candies, and chocolates), bread, and cereals. In the case of food for specified health use, nutritional supplement food, functional food, or the like, the composition may be in the form of capsules, troches, syrup, granules, powder, or the like.

### EXAMPLES

### Example 1

Euglena gracilis powder (from Euglena Co., Ltd.) was used as Euglena of Example 1.

### Example 2

Crystalline paramylon was prepared in the following manner.

The Euglena gracilis powder of Example 1 (from Euglena Co., Ltd.) was added to distilled water and stirred at room temperature for 2 days. The resultant was ultrasonically treated to destroy the cell membranes, and the crude paramylon particles were collected by centrifugation. The collected paramylon particles were dispersed in a 1% aqueous solution of sodium dodecyl sulfate and treated at 95° C. for 2 hours. After the paramylon particles were collected by centrifugation, the particles were dispersed in a 0.1% aqueous solution of sodium dodecyl sulfate and treated at 50° C. for 30 minutes. The lipid and the proteins were removed by these operations. Then, the remainder was washed with acetone and ether and dried at 50° C. to give purified paramylon particles.

1 g of the prepared paramylon was enclosed in a known capsule to prepare an immune balance regulator of Example 2.

### Example 3

The paramylon prepared in Example 2 was used to prepare amorphous paramylon according to a method described in Japanese Patent No. 5612875.

In particular, the crystalline paramylon powder prepared in Example 2 was added to and dissolved in 1 N aqueous sodium hydroxide at a concentration of 5% (w/v) and stirred for 1-2 hours with a stirrer for alkali treatment. Then, 1 N hydrochloric acid was added dropwise to the solution of the paramylon powder in the 1N aqueous sodium hydroxide to neutralize the solution. After centrifugation, the supernatant was removed, and the precipitate was repeatedly washed with distilled water. Then, the precipitated gel was collected. After freezing, the gel was lyophilized using a lyophilizer to give amorphous paramylon of Example 3.

### Example 4

The paramylon prepared in Example 2 was used to prepare emulsion paramylon in the following manner.

Ion-exchanged water was added to crystalline paramylon powder (from Euglena Co., Ltd., median size: 2.591 μm) to give a paramylon slurry having a paramylon concentration of 10 wt %.

A liquid medium in a wet pulverizer (Start Burst 18KW, Mid-Scale, Sugino Machine Ltd., Oblique Collision Chamber) was exchanged for ion-exchanged water. The nozzle of the pulverizer is pressurized, and the paramylon slurry is fed into the pulverizer. The initially discharged liquid was discarded as the dead volume in the pulverizer. Then, oblique jet collision was produced by ejecting jets of the paramylon slurry from a pair of nozzles opposed to each other at an angle to cause the slurry to collide with each other. The treated slurry was collected from the outflow channel, which was regarded as 1 pass. The treatment pressure was 245 MPa, the amount of slurry treated was 240 mL, and the diameter of the nozzles was 0.17 mm.

The above treatments were repeated 3 times (3 passes) to give emulsion paramylon of Example 4.

The emulsion paramylon of Example 4 was not separated from the ion-exchanged water added in preparation of the slurry and was swelled by binding to water. The emulsion paramylon of Example 4 had a median size of 27.127 μm (as measured on LA-960 laser diffraction/scattering particle size distribution analyzer from Horiba, Ltd.).

### Study Example 1

### Study of Administration of Immune Balance Regulator to Healthy Early Elderly Persons

The immune balance regulator of Example 2 was used to conduct a human clinical trial of the effect of regulating the immune balance by continuous administration of the immune balance regulator for 8 weeks.

10 healthy early elderly persons, aged 60-65, (5 males and 5 females) participated in the study. Their average age was 62.80, and their average body weight was 58.93 kg at the beginning of the study.

A capsule of the immune balance regulator of Example 2 was administered to the participants once a day every day after meals. Note that the regulator could be administered at any time of the day. The regulator was continuously administered for 8 weeks.

Immediately before the study (week 0) and 4 and 8 weeks after the initiation of the study, a blood sample was collected from each of the participants. The collected whole blood samples were used to determine the amount of various cytokines in the culture supernatant stimulated with PMA (Phorbol 12-myristate 13-acetate) and Ionomycin by a known method.

In particular, 8 mL of the peripheral blood was collected into a collection tube for isolation of mononucleosis (Becton Dickinson, 362761) and then centrifuged at 3000 rpm for 20 minutes. Then, the cell layer on the gel barrier was collected into a 50 mL tube. 30 mL of saline was added to the collected cells and centrifuged at 1500 rpm for 10 minutes. The supernatant was removed, and 10 mL of saline was added. Then, the resultant was centrifuged at 1200 rpm for 5 minutes and suspended in RPMI-1640 cell culture medium (Gibco, 11875-093). 1×10⁶ monocytes were cultured in cell culture medium supplemented with 10% (final concentration) FBS, 50 ng/mL of LPMA (Phorbol 12-Myristate 13-Acetate, Sigma, P1585), and 500 ng/mL of Ionomycin (Sigma, 19657) for 48 hours, and the culture supernatant was collected. The culture supernatant was stored at −80° C. until measurement. The amount of cytokines in the culture supernatant was quantitatively analyzed according to the procedure for a cytokine measurement kit (Flowcytomix, eBioscience, BMS810FFRTU).

The content of the monocytes was also determined. In particular, the peripheral blood was collected from each of the participants into a collection tube with EDTA-2K and analyzed on a flow cytometer (Beckman-Coluter, Navios) using various fluorescently-tagged antibodies. In the analysis, data for lymphocyte populations positive for FSC, SSC, and CD45 antibodies was acquired and analyzed.

The following combination of antibodies were used (all of the antibodies were obtained from Beckman Coluter, Inc.).

(1) PC7-labeled anti-CD45 antibodies, PE-labeled anti-CD3 antibodies, FITC-labeled anti-CD20 antibodies, APC-labeled anti-CD56 antibodies, and PC5-labeled anti-CD16 antibodies

(2) PC7-labeled anti-CD45 antibodies, FITC-labeled anti-CD8 antibodies, APC-labeled anti-CD4 antibodies, PC5-labeled anti-CD28 antibodies, and ECD-labeled anti-CD45RA antibodies

The study results are illustrated in FIGS. 1-8.

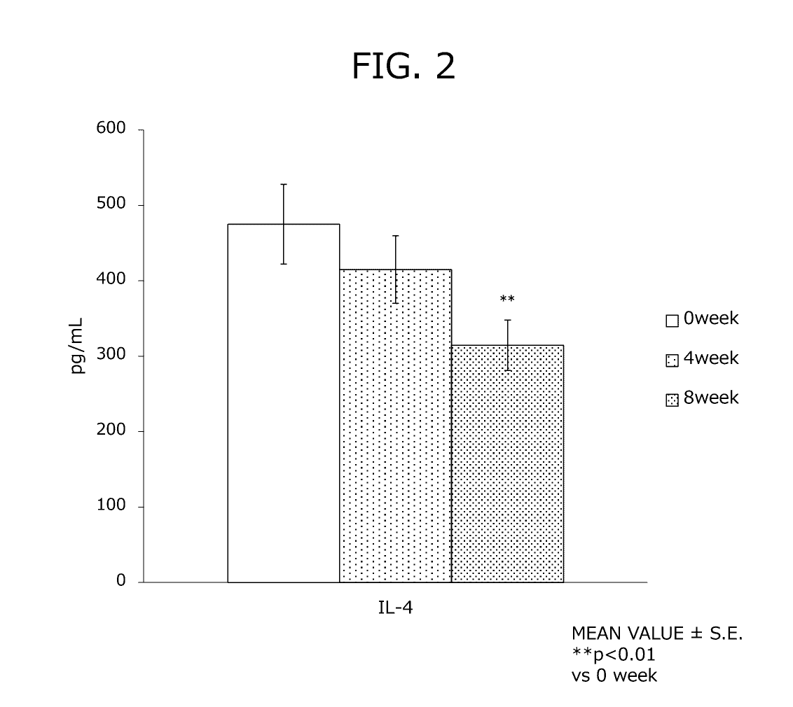

FIGS. 1 and 2 indicate that IFN-γ was increased significantly (p<0.05 by t-test), and IL-4 was reduced significantly (p<0.01 by t-test) in the 8 week administration period.

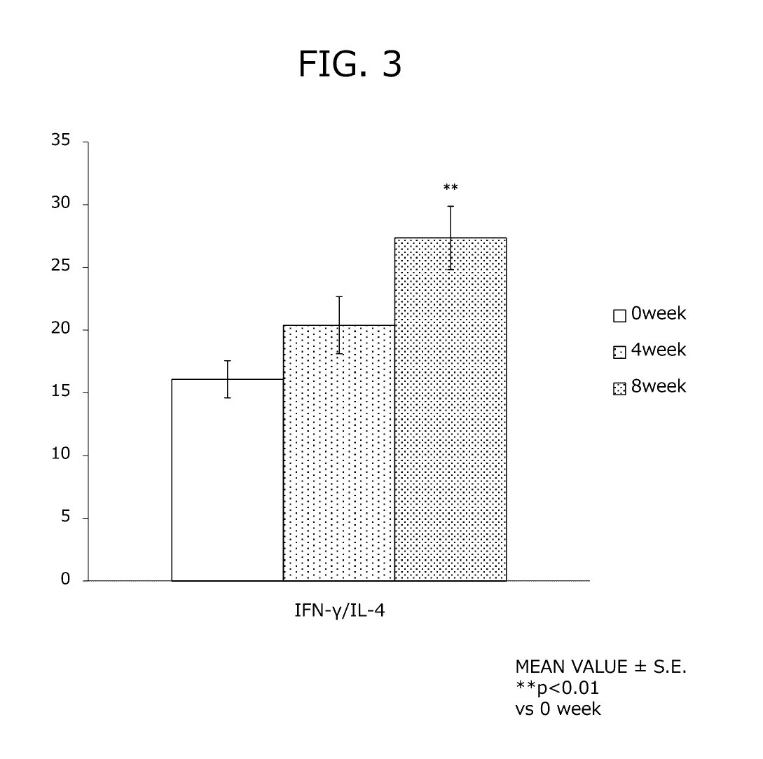

The data in FIGS. 1 and 2 were used to calculate the ratio of the IFN-γ production to the IL-4 production, i.e., IFN-γ/IL-4, which was increased significantly (p<0.01 by t-test), as illustrated in FIG. 3. Thus, it has been found that the humoral immunity was greater than the cellular immunity, and that the 8 week administration increased the preponderance of the cellular immunity (immune responses induced by Th1) over the humoral immunity (immune responses induced by Th2).

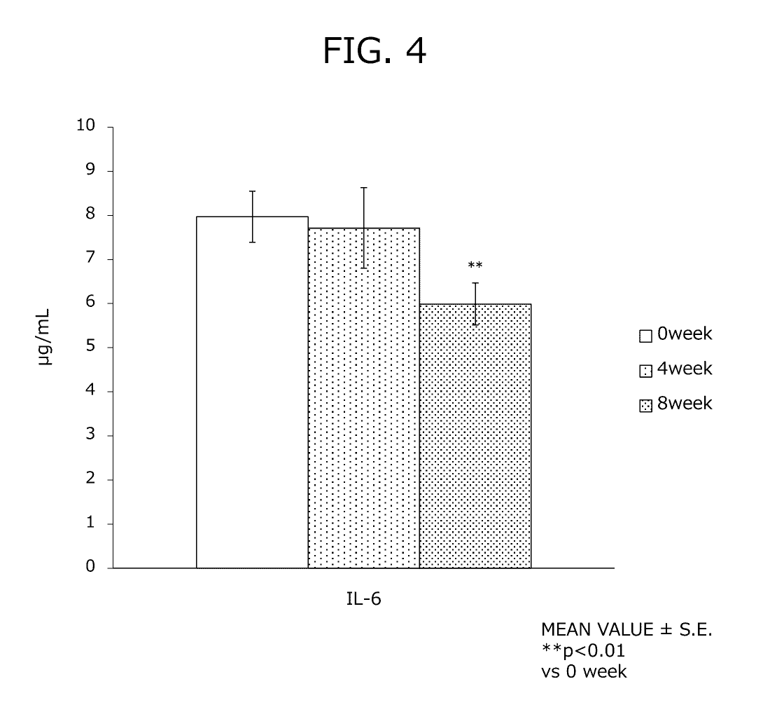

As illustrated in FIG. 4, IL-6, which promotes the differentiation of Th0 into Th2, were reduced significantly (p<0.01 by t-test) and were reduced over time in the 8 week administration period.

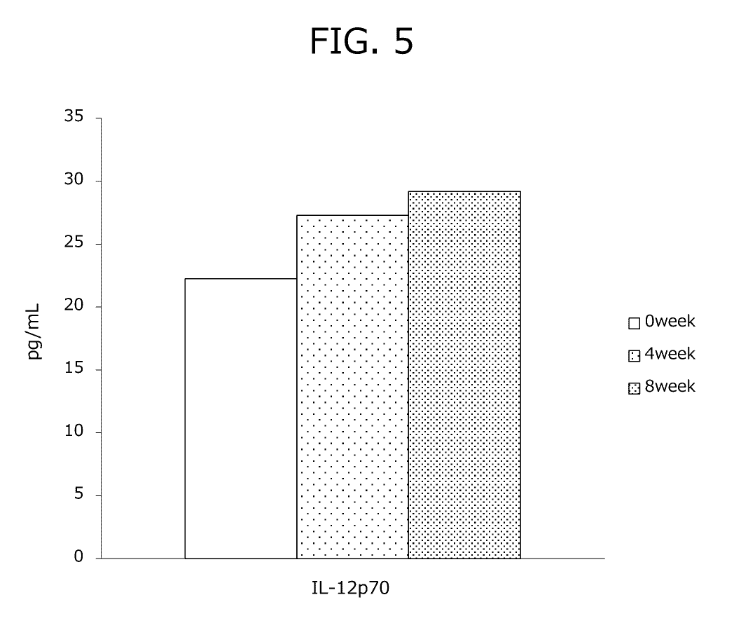

In contrast, as illustrated in FIG. 5, IL-12, which promotes the differentiation of Th0 into Th1, tended to be increased over time in the 8 week administration period.

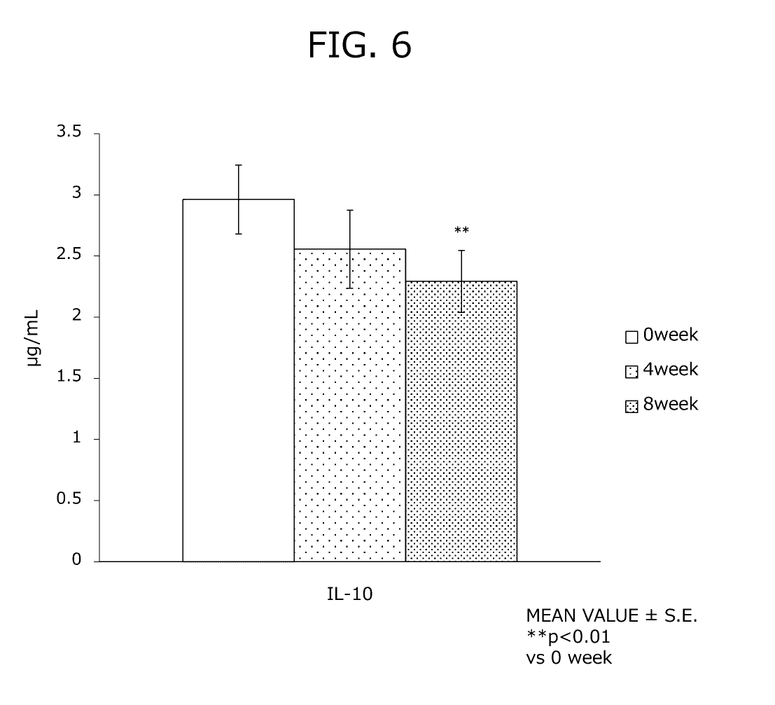

As illustrated in FIG. 6, IL-10, which inhibits the production of IFN-γ and IL-12, was reduced significantly (p<0.01 by t-test) and were reduced over time in the 8 week administration period.

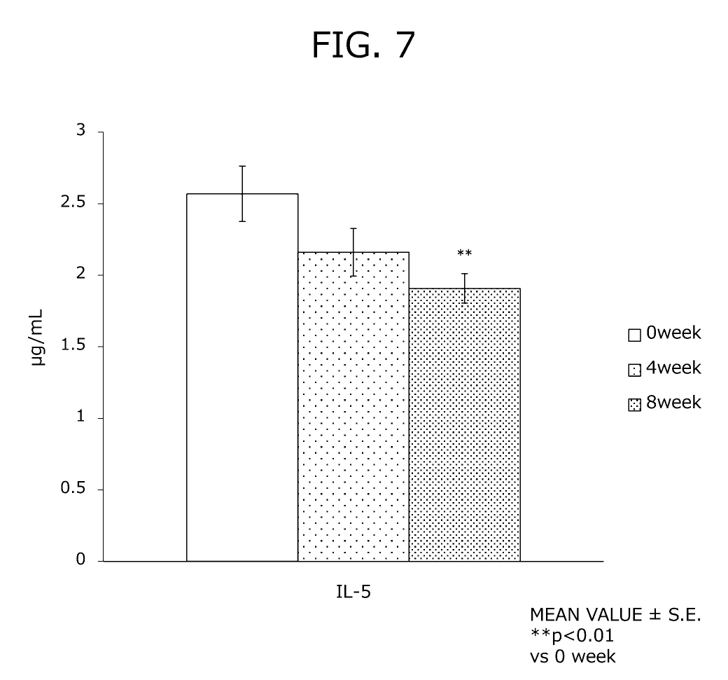

As illustrated in FIG. 7, IL-5, which induces the proliferation and the differentiation of B cells and is involved in humoral immunity, was reduced significantly (p<0.01 by t-test) and were reduced over time in the 8 week administration period.

The results in FIGS. 1-7 indicate that IFN-γ, which are involved in cellular immunity, were increased significantly, and IL-12 tended to be increased, while IL-4, IL-5, IL-6, and IL-10, which are involved in humoral immunity, were reduced significantly.

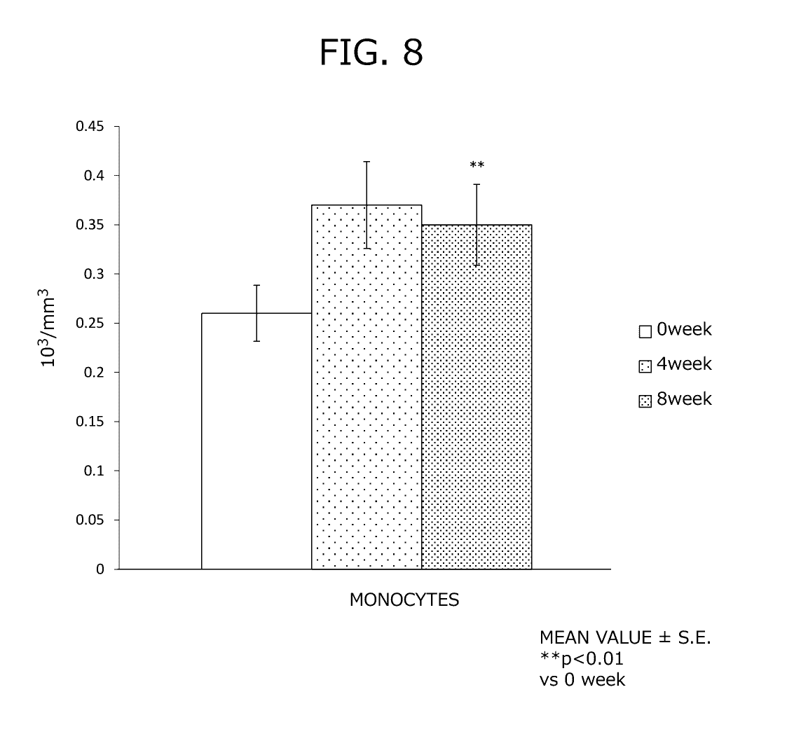

The results of the study on monocytes are illustrated in FIG. 8.

The results in FIG. 8 indicate that monocytes, which play an important role in the initiation of immunity against infection and of which production is promoted by cellular immunity, were significantly increased.

### Discussion of Study Example 1

The results of Study Example 1 indicate that administration of the immune balance regulator of Example 2 to the healthy early elderly persons, aged 60-65, for 8 weeks significantly increased IFN-γ, which promotes the differentiation into Th1, and created the tendency for IL-12, which promotes the differentiation into Th1, to be increased.

IL-4, which promotes the differentiation into Th2, induces the activation and the proliferation of B cells, inhibits promotion of the differentiation into Th1, induces the proliferation of Th2, and inhibits the activation of macrophages; IL-5, which induces the proliferation and the differentiation of B cells; and IL-10, which inhibits the production of INF-γ and IL-12, was reduced significantly.

Thus, it has been found that the immune balance regulator of Example 2 inhibits the production of IL-4, IL-5, and IL-10 cytokines, which activate humoral immunity and inhibit activity of cellular immunity; promotes the production of IFN-γ, which activates cellular immunity; and promotes the production of monocytes, which play an important role in the initiation of immunity against infection, thereby regulating the immune balance in the healthy early elderly persons, aged 60-65.

The following Study Examples 2-5 confirmed the anti-viral effect, the effect of inhibiting an infection upon infection with a virus, and the effect of alleviating influenza symptoms provided by administration of Euglena, paramylon, and amorphous paramylon.

### Study Example 2

### Study of Survival Rates of Mice Infected with Influenza Virus

Mice that have taken the paramylon prepared in Example 2, the amorphous paramylon of Example 3, or the Euglena gracilis powder of Example 1 (from Euglena Co., Ltd.) were infected with influenza virus to conduct the study of the anti-influenza effect of paramylon, amorphous paramylon, and Euglena.

In the study, BALB/c Cr Slc (SPF) male mice (Japan SLC, Inc.) were used. Diets and water (distilled water) were available ad libitum.

The mice were classified into a control group, a paramylon group, an amorphous paramylon group, and a Euglena group.

The study was conducted twice. In the first study, the paramylon prepared in Example 2, the amorphous paramylon of Example 3, and the Euglena gracilis powder of Example 1 (from Euglena Co., Ltd.) were respectively added, at a concentration of 2%, to diets for the paramylon group, the amorphous paramylon group, and the Euglena group for a week prior to infection with the virus. In the second study, the respective regulator was added to the diets in the similar manner for 2 weeks prior to infection with the virus.

In the first study, the number of mice (n) per group was 7, while in the second study, n per group was 15.

Then, in both of the first and second studies, 1000 PFU of Influenza virus A/PR/8/34 (H1N1) was intranasally administered to 6 week old mice in each of the groups for intranasal infection. Then, the mice in each of the groups were inspected for survival for 10 days after the infection.

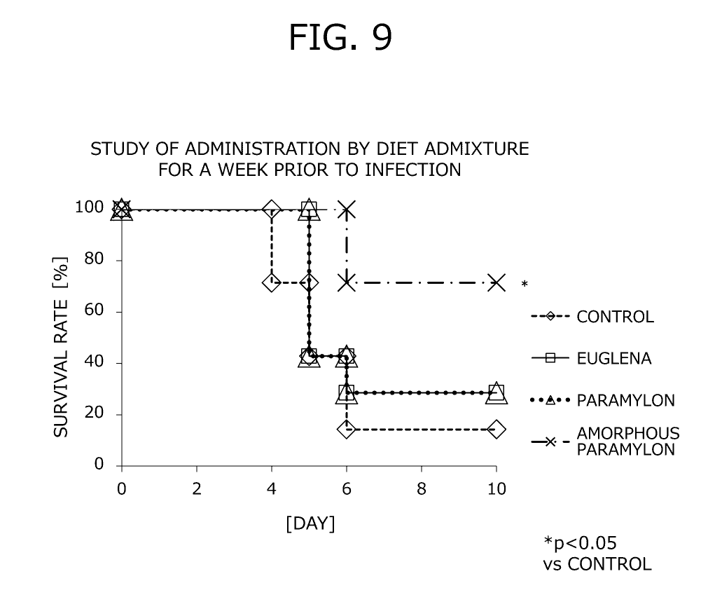

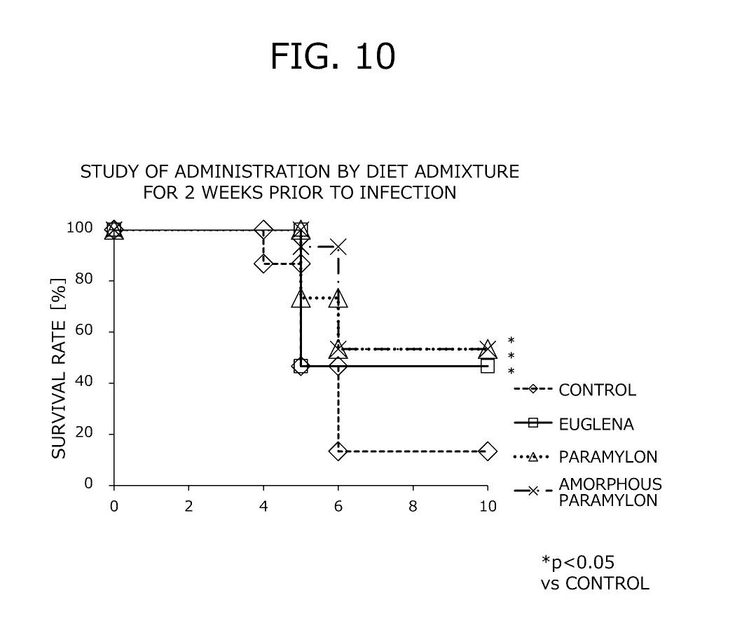

The results of the first study are illustrated in FIG. 9, while the results of the second study are illustrated in FIG. 10. In the first study of administration for a week prior to the infection in FIG. 9, a chi-squared test showed a significant difference between the control group and the amorphous paramylon group at day 7 after the infection (p=0.0308).

A chi-squared test showed a significant difference between the control group and the Euglena group at day 10 after the infection (p=0.0464). There also was a significant difference among the control group, the paramylon group, and the amorphous paramylon group at day 10 after the infection (p=0.0201).

The Study Example has revealed that oral administration of the amorphous paramylon of Example 3 for a week prior to the infection with influenza virus significantly inhibits death caused by the infection with influenza virus.

It has been found that oral administration of the paramylon of Example 2, the amorphous paramylon of Example 3, or the Euglena of Example 1 for 2 weeks prior to the infection with influenza virus significantly inhibits death caused by the infection with influenza virus. As oral administration of the paramylon, the amorphous paramylon, or the Euglena improved the functions of the cellular immunity induced by Th1, development of an influenza disease was inhibited, or the symptoms of a developed influenza disease were weakened, when the mice were infected with an influenza virus after improvement of the functions of the cellular immunity.

### Study Example 3

### Measurement of Virus Titers

In the same manner as in the first study and the second study in Study Example 2 in which the diet admixtures were administered to the mice for a week and 2 weeks prior to infection respectively, the mice of each of the groups (n=3) were intranasally infected with Influenza virus A/PR/8/34 (H1N1) and determined for virus titers in the lungs at day 2 after the infection.

For measurement of the virus titers, first, the lungs were removed from the mice of each of the groups at day 2 after the infection, and lung homogenates were made by homogenizing the removed lungs in 1 mL of PBS (−).

Then, the virus titers are measured by plaque titration. In particular, a day before the infection with the virus, MDCK cells suspended in E's MEM medium (Eagle's MEM medium “Nissui” (E's MEM), Nissui Pharmaceutical Co., Ltd.) supplemented with 10% FBS (fetal bovine serum) were plated on a 6 well plate at 5×10⁵ cells/well and cultured in monolayer overnight under 5% CO₂ at 37° C. The MDCK cell monolayers were used in the study.

After the MDCK cells were washed with the E's MEM, 500 μL of the lung homogenate serially diluted 10-fold in the E's MEM was inoculated. After adsorption under 5% CO₂ at 35° C. for an hour, the virus was removed. 2 mL of 0.75% Agarose 1600 (Wako Pure Chemical Industries, Ltd.) heated to 43° C., 0.0015% DEAE-dextran (Pharmacia Biotech), and 3 μg/mL of E's MEM supplemented with acetyl trypsin (SIGMA, T-6763) were overlayed onto each of the wells, which then were incubated at room temperature until the overlay had completely solidified. After the solidification, the plate was cultured under 5% CO₂ at 35° C. for 3 days. After completion of the cultivation, the cultures were fixed in 10% formalin. After the fixation, the medium was removed, and the cells were stained with 0.5% amido black.

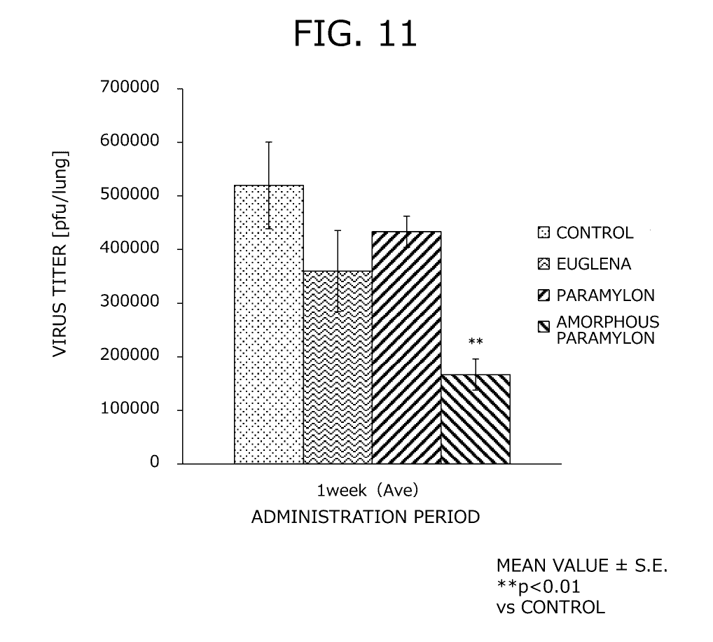

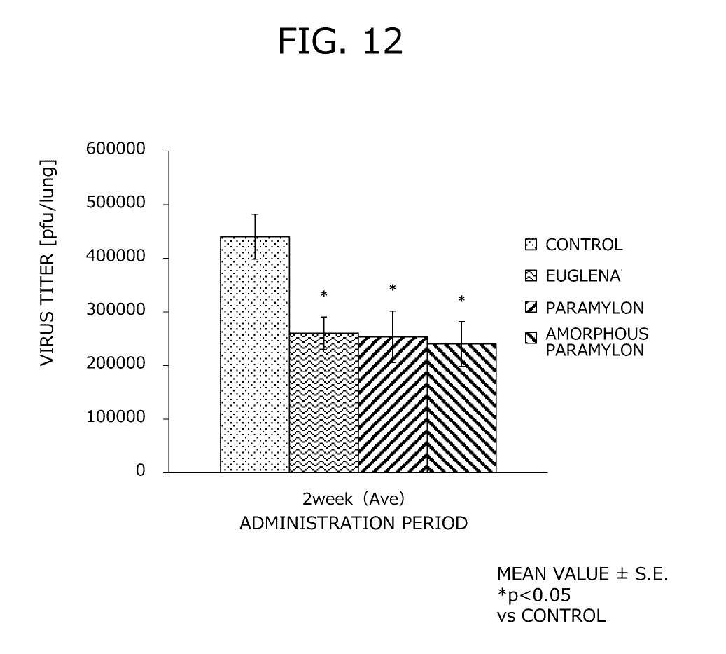

The results are illustrated in Table 1 and FIGS. 11 and 12.

TABLE 1

| | | | | |
|---|---|---|---|---|
| | | | | Amorphous |
| Mouse No. | Control | Euglena | Paramylon | Paramylon |

1 Week Administration (Day 2 after Infection) (Unit: PUF/lung)

| | | | | |
|---|---|---|---|---|
| #1 | 620000 | 380000 | 480000 | 220000 |
| #2 | 360000 | 220000 | 380000 | 120000 |
| #3 | 580000 | 480000 | 440000 | 160000 |
| Ave | 520000 | 360000 | 433333 | 166667 |
| SE | 80829 | 75719 | 29059 | 29059 |

2 Week Administration (Day 2 after Infection) (Unit: PUF/lung)

| | | | | |
|---|---|---|---|---|
| #1 | 380000 | 320000 | 160000 | 220000 |
| #2 | 520000 | 240000 | 280000 | 180000 |
| #3 | 420000 | 220000 | 320000 | 320000 |
| Ave | 440000 | 260000 | 253333 | 240000 |
| SE | 41633 | 30551 | 48074 | 41633 |

The results in Table 1 and FIGS. 11 and 12 reveal that the 1-week Euglena group, the 1-week paramylon group, the 1-week amorphous paramylon group, the 2-week Euglena group, the 2-week paramylon group, and the 2-week amorphous paramylon group respectively showed 69.2%, 83.3%, 32.0%, 59.1%, 57.6%, and 54.5% reduction in virus titer and thus administration of Euglena, paramylon, or amorphous paramylon prior to infection with influenza virus significantly inhibits influenza infection as determined by Dunnett's test.

### Study Example 4

### Measurement of Virus Titers

The mice of each of the groups (n=5) were intranasally infected with Influenza virus A/PR/8/34(H1N1) in the same manner as in the study of administration of the diet admixture for 2 weeks prior to the infection in Study Example 2 and were determined for virus titers in the lungs of the mice of each of the groups at days 1, 2, and 3 after the infection in the same manner as in Study Example 3.

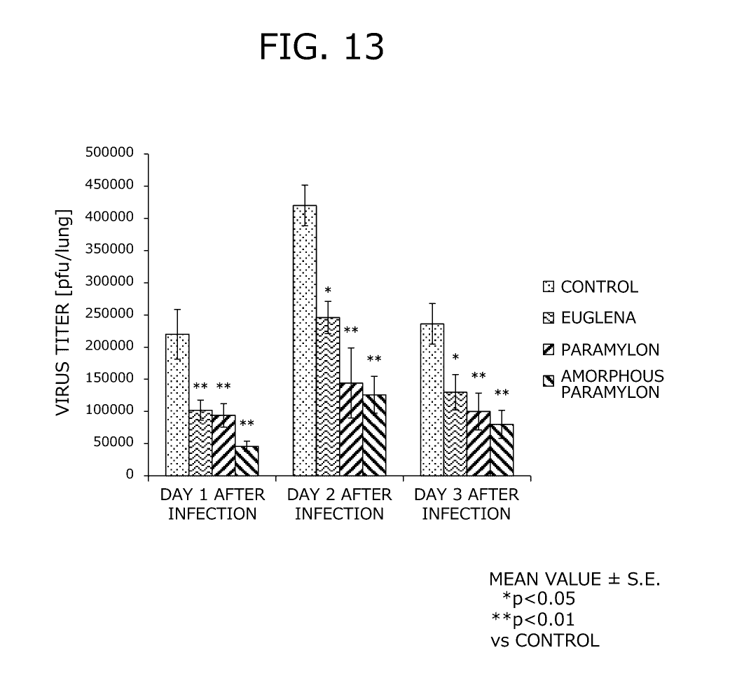

The results are illustrated in Table 2 and FIG. 13.

TABLE 2

| | | | | |
|---|---|---|---|---|
| | | | | Amorphous |
| Mouse No. | Control | Euglena | Paramylon | Paramylon |

Day 1 after Infection (Unit: PUF/lung)

| | | | | |
|---|---|---|---|---|
| #1 | 230000 | 80000 | 120000 | 30000 |
| #2 | 200000 | 100000 | 150000 | 60000 |
| #3 | 360000 | 60000 | 70000 | 40000 |
| #4 | 180000 | 120000 | 80000 | 70000 |
| #5 | 130000 | 150000 | 50000 | 30000 |
| Ave | 220000 | 102000 | 94000 | 46000 |
| SE | 38601 | 15620 | 18055 | 8124 |

Day 2 after Infection (Unit: PUF/lung)

| | | | | |
|---|---|---|---|---|
| #1 | 510000 | 220000 | 15000 | 120000 |
| #2 | 430000 | 340000 | 26000 | 230000 |
| #3 | 390000 | 190000 | 160000 | 130000 |
| #4 | 320000 | 240000 | 290000 | 90000 |
| #5 | 450000 | 240000 | 230000 | 60000 |
| Ave | 420000 | 246000 | 144200 | 126000 |
| SE | 31623 | 25219 | 54559 | 28740 |

Day 3 after Infection (Unit: PUF/lung)

| | | | | |
|---|---|---|---|---|
| #1 | 190000 | 50000 | 50000 | 50000 |
| #2 | 310000 | 130000 | 180000 | 160000 |
| #3 | 290000 | 220000 | 150000 | 60000 |
| #4 | 250000 | 110000 | 90000 | 40000 |
| #5 | 140000 | 140000 | 30000 | 90000 |
| Ave | 236000 | 130000 | 100000 | 80000 |
| SE | 31559 | 27386 | 28636 | 21679 |

The results in Table 2 and FIG. 13 reveal that the day 1 Euglena group, the day 1 paramylon group, the day 1 amorphous paramylon group, the day 2 Euglena group, the day 2 paramylon group, the day 2 amorphous paramylon group, the day 3 Euglena group, the day 3 paramylon group, and the day 3 amorphous paramylon group showed 46.4%, 42.7%, 20.9%, 58.6%, 34.3%, 30.0%, 55.1%, 42.4%, and 33.9% reduction in virus titer, and thus administration of Euglena, paramylon, or amorphous paramylon prior to infection with influenza virus significantly inhibits influenza infection as determined by Dunnett's test.

### Study Example 5

### Measurement of Cytokines in Lungs in Mice Infected with Influenza Virus

The mice that took the paramylon prepared in Example 2, the amorphous paramylon of Example 3, or the Euglena gracilis powder of Example 1 (from Euglena Co., Ltd.) were infected with influenza virus to measure cytokines (IL-113, IL-6, IL-10, IL-12 (p70), IFN-γ, TNF-α, and IFN-β) in the lungs.

After 4 week old BALB/c Cr Slc (SPF) male mice (Japan SLC, Inc.) were acclimated for a week and were given a purified diet (control group) or the purified diet admixed with the Euglena gracilis powder of Example 1 (Euglena group), the paramylon prepared in Example 2 (paramylon group), or the amorphous paramylon of Example 3 (amorphous paramylon group) at 2% ad libitum from week 2 prior to virus inoculation to a laparotomy. 6 week old mice that took the test material from 4 weeks of age for 2 weeks were intranasally inoculated with an LD50 dose (1000 PFU) of influenza virus A/PR/8/34 (H1N1).

At days 1, 2, and 3 after the virus inoculation, a laparotomy was performed to remove the lungs. The lungs were homogenized to measure various cytokines. The measurement was performed by ELISA for IFN-β and by Bio-Plex for other cytokines.

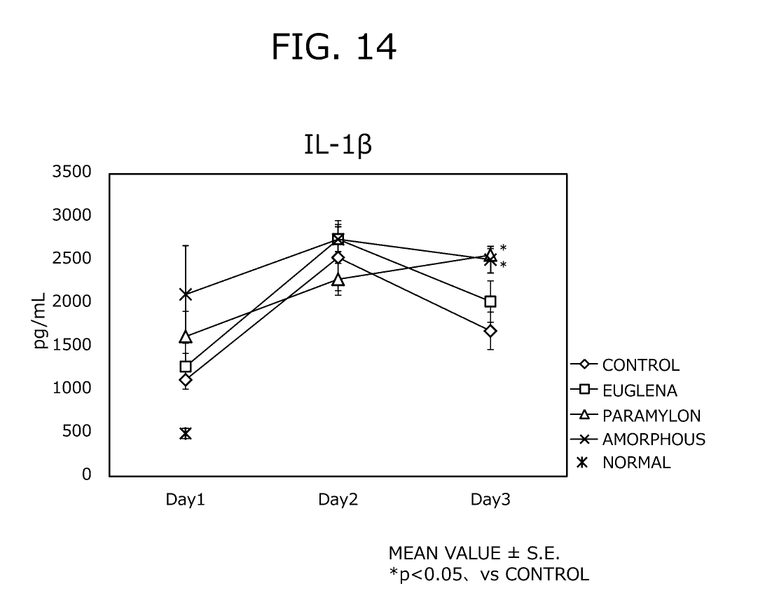

The measurements of each of the cytokines were illustrated in FIGS. 14-20. Note that FIGS. 14-20 illustrate, as data for a normal group, measurements of each of the cytokines for mice that were housed for 2 weeks in the same manner as the control group, that were not inoculated with virus, and that were subjected to a laparotomy at a day corresponding to day 1 after the virus inoculation of the other groups.

With regard to the cytokines present in the lungs at days 1, 2, and 3 after the virus inoculation, the results in FIGS. 14-20 indicate that the paramylon group and the amorphous paramylon group exhibited a significantly large amount of IL-6 and TNF-α inflammatory cytokines at day 1 after the virus inoculation. At day 3 after the virus inoculation, the paramylon group and the amorphous paramylon group exhibited a significantly larger amount of IL-10. The results suggest that release of the inflammatory cytokines early after infection leads to defense against infection, provided by inflammation, which is subsequently inhibited by release of IL-10, thereby contributing to increased survival rate.

And at day 1 after the infection, the paramylon group and the amorphous paramylon group exhibited a significantly larger amount of IL-12. Then, the paramylon group and the amorphous paramylon group exhibited a significantly larger amount of IFN-γ.

The results have revealed a mechanism of action in which the production of IL-12 activates NK cells, which induce IFN-γ.

At day 2, the amorphous paramylon group exhibited a significantly larger amount of IFN-β, which is a cytokine with anti-viral action.

FIGS. 14-20 indicate that the Euglena group, the paramylon group, and the amorphous paramylon group had different behaviors. The behavior difference suggests that the Euglena contains a relatively small amount of paramylon and thus had a less effect, which has revealed that paramylon is an immunologically active ingredient.

Typically, mice infected with influenza die due to strong lung inflammation. The results of Study Examples 2-5 indicate that the mice of the Euglena group, the paramylon group, and the amorphous paramylon group exhibited a significantly higher survival rate after infection with influenza virus and lower virus titers compared with those of the control group. It is expected that in the mice of the Euglena group, the paramylon group, and the amorphous paramylon group, inflammatory cytokines were released early after infection to provide defense against infection, and then the inflammation caused in the process was inhibited by release of IL-10, which led to increased survival rate and decreased virus titers in the lungs.

Thus, it has been found that Euglena, paramylon, and amorphous paramylon have the effect of alleviating influenza symptoms.

### Study Example 6

### Study of Pharmacological Actions in Gastric Ulcer Models

In a water immersion stress test in rats, the Euglena of Example 1, the paramylon of Example 2, and the amorphous paramylon of Example 3 were administered, and the inhibition effect of Examples 1-3 on a gastric ulcer, which is an exemplary stress disease.

6 week old male rats (Wistar) were pre-conditioned on a conditioning diet (CLEA Rodent Diet CE-2, CLEA Japan, Inc.) for 4 days prior to the initiation of the study, and then rats of the control group, the Example 1 Euglena group, the Example 2 paramylon group, the Example 3 amorphous paramylon group were fed on the diets described in Table 3 for 14 days.

In Table 3, the diet for the Example 1 group was prepared by reducing the amount of the respective ingredients of the diet for the control group to 97% and adding Euglena in an amount of 3% based on the total weight. The diets for the Example 2 and 3 groups were prepared by reducing the amount of cellulose in the diet for the control group by 3% and adding paramylon or amorphous paramylon in an amount of 3%. As paramylon and amorphous paramylon are a glucan, paramylon and amorphous can be nutritionally substitute for cellulose. In contrast, as Euglena contains not only glucan, but also various nutrients, Euglena substitutes for 3% of the respective ingredients.

Thus, the diets for the respective groups have an energy ratio and an energy density of the three major nutrients as illustrated in Table 4 and have the substantially same nutrient balance.

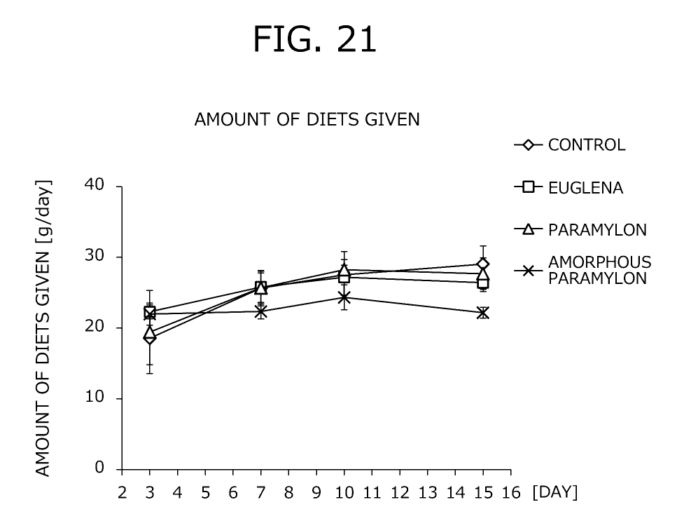

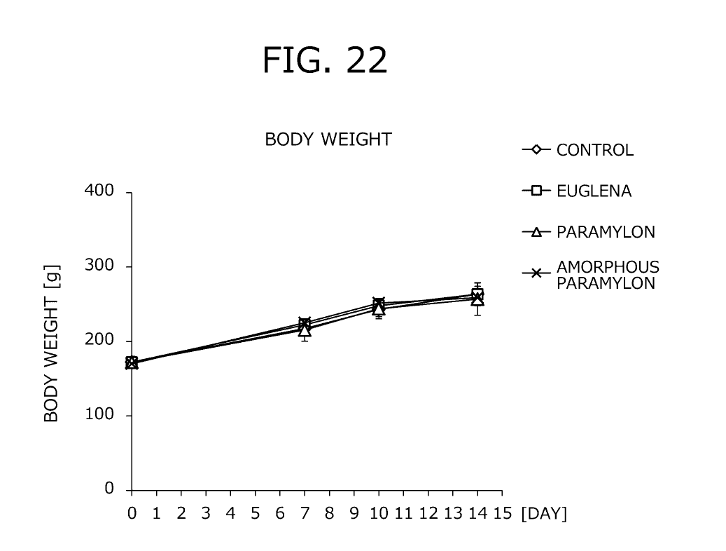

The amounts of the diets given to the rats of the respective groups for 14 days are illustrated in FIG. 21, and the body weights are illustrated in FIG. 22.

TABLE 3

| | | | | Amorphous |
|---|---|---|---|---|
| | | Euglena | Paramylon | Paramylon |
| Composition | Control | Group | Group | Group |
| (%) | Group | (Example 1) | (Example 2) | (Example 3) |
| Casein | 14.0 | 13.6 | 14.0 | 14.0 |
| L-cystine | 0.18 | 0.17 | 0.18 | 0.18 |
| β-cornstarch | 46.6 | 45.2 | 46.6 | 46.6 |
| α-cornstarch | 15.5 | 15.0 | 15.5 | 15.5 |
| Sucrose | 10.0 | 9.7 | 10.0 | 10.0 |
| Soybean Oil | 4.0 | 3.9 | 4.0 | 4.0 |
| Cellulose | 5.0 | 4.9 | 2.0 | 2.0 |
| Mineral Mix | 3.5 | 3.4 | 3.5 | 3.5 |
| (AIN-93M-MX) | | | | |
| Vitamin Mix | 1.0 | 1.0 | 1.0 | 1.0 |
| (AIN-93V-MX) | | | | |
| Choline | 0.25 | 0.24 | 0.25 | 0.25 |
| Bitartrate | | | | |
| TBHQ | 0.0008 | 0.0008 | 0.0008 | 0.0008 |
| Sample of | 0 | 3 | 3 | 3 |
| Example 1, 2, | | | | |
| or 3 | | | | |
| Total (%) | 100 | 100 | 100 | 100 |

TABLE 4

| | | | | Amorphous |
|---|---|---|---|---|
| | | Euglena | Paramylon | Paramylon |
| Total Energy | Control | Group | Group | Group |
| (%) | Group | (Example 1) | (Example 2) | (Example 3) |
| Protein | 0.49 | 0.55 | 0.49 | 0.49 |
| Carbohydrate | 2.61 | 2.55 | 2.61 | 2.61 |
| Fat | 0.42 | 0.41 | 0.42 | 0.42 |
| Energy | 3.52 | 3.51 | 3.52 | 3.52 |
| (kcal/g | | | | |
| diet) | | | | |

The rats of each of the groups were given the diets described in Table 3 for 14 days and then were fasted overnight.

Then, the rats of each of the groups were restrained in a stress cage for 18 hours and immersed in water to chest level. Then, the rats were dissected to examine a gastric ulcer.

After the rats of each of the groups were weighed, the kidneys, spleens, duodenums, and epididymal adipose tissues were removed and weighed in the groups, and the weights of the respective organs were compared with the body weight of the rats to determine the relative weights. Then, comparison of the results with the weights of the control group indicated that the organs except for the duodenums exhibited no change in relative weight. In contrast, only the duodenums in the Euglena group (Example 1) and the paramylon group (Example 2) exhibited a significant increase (p<0.05 by Tukey-Kramer test). Thus, it is expected that the present invention provides the effect of growing digestive organs. The relative weights of the duodenums are illustrated in Table 5.

TABLE 5

| | Diet Group | | | |
|---|---|---|---|---|
| | | | | Amorphous |
| | | Euglena | Paramylon | Paramylon |
| Relative | Control | Group | Group | Group |
| Weight (g %) | Group | (Example 1) | (Example 2) | (Example 3) |
| Duodenums | 0.085 ± 0.0052^{a} | 0.110 ± 0.0054^{b} | 0.118 ± 0.0063^{b} | 0.100 ± 0.0096^{a} |

Mean ± SD (g %)
^{ab}p <0.05

Stomachs in each of the groups were removed, and ulcers in the mucosal surfaces were photographed and measured.

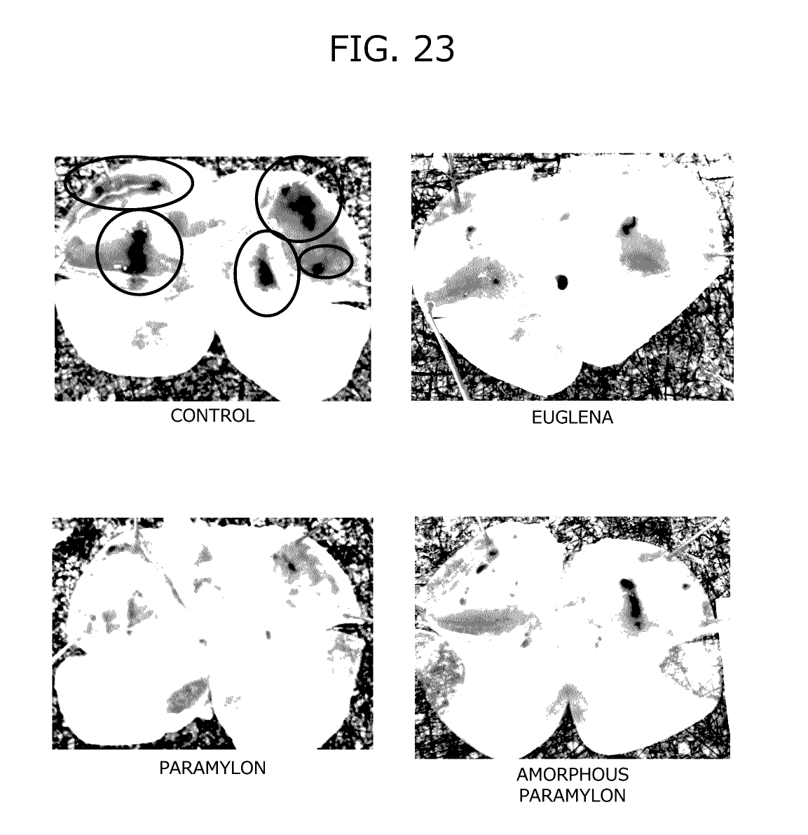

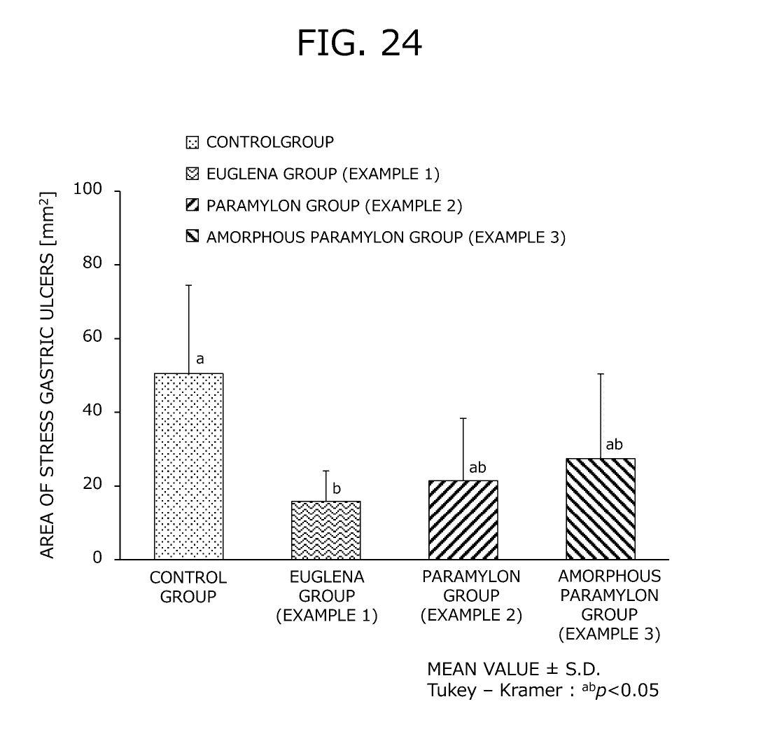

The photographs of representative gastric ulcers in each of the groups are illustrated in FIG. 23, and the areas of the ulcers are illustrated in FIG. 24.

As illustrated in FIG. 23, the gastric ulcer portions (within ellipses) that became black due to blood stain were clearly observed in the control group, while gastric ulcer portions markedly shrunk in the Example 1-3 groups (the Euglena group, the paramylon group, and the amorphous paramylon group), compared with the control group. Particularly, gastric ulcer portions markedly shrunk in the Euglena group (Example 1) and the paramylon group (Example 2).

As illustrated in FIG. 24, the Euglena group (Example 1) exhibited a significantly smaller area of gastric ulcers compared with the control group (p<0.05 by Tukey-Kramer test). The paramylon group (Example 2) and the amorphous paramylon group (Example 3) also tended to exhibit a smaller area. As illustrated in Table 5, the Euglena group (Example 1) and the paramylon group (Example 2) exhibited an increased relative weight of duodenums. Thus, it is expected that the regulators have a mechanism of action for protecting digestive organs against stress.

In a similar water immersion stress test in rats, rats that were given the diets in admixture with the Euglena of Example 1, the paramylon of Example 2, or the amorphous paramylon of Example 3 in Table 3 and the rats that were given the control diet in Table 3 were immersed in water to chest level for 3.5 hours and dissected in the similar manner. Then, gastric mucosae of the rats of the control group and the Example 1-3 groups were removed and amplified using RT-PCR (using T100™ Thermal Cycler (BIO-RAD) System). The PCR products were analyzed on 2% agarose gel to examine expression of iNOS (inducible nitric oxide synthase) and expression of COX-2 (inducible cyclooxygenase).

As used herein, iNOS is a type of nitric oxide synthases (NOS), which produce nitric oxide from L-arginine and oxygen via an oxidation reaction. NOS are classified into neuronal NOS (type I, neuronal NOS 1, nNOS), endothelial NOS (type III, endothelial NOS, eNOS), and inducible NOS (type II, iNOS). iNOS naturally binds calmodulin and calcium and does not require increase in intracellular free calcium. iNOS is induced by cytokines and intracellular toxins and known to be involved in inflammatory conditions. Nitric oxide derived from iNOS has anti-virus and anti-bacterial effects in the host defense system and plays an important role in defense against infection, while the nitric oxide also leads to excessive inflammation (Med. Bull, Fukuoka Univ., 29(4), 247-255, 2002).

COX-2 is a type of cyclooxygenases (COX). COX are rate-limiting enzymes in biosynthesis of prostaglandin (PG) and have two isozymes: COX-1 and COX-2. COX-2 is an inducible enzyme and is involved in conditions such as inflammation and oncogenesis and mainly exists in nuclear membranes in cells. COX expressed at a site of inflammation are mainly COX-2, and expression of COX-2 at a site of inflammation induces synthesis of PG, which then leads to excessive inflammation.

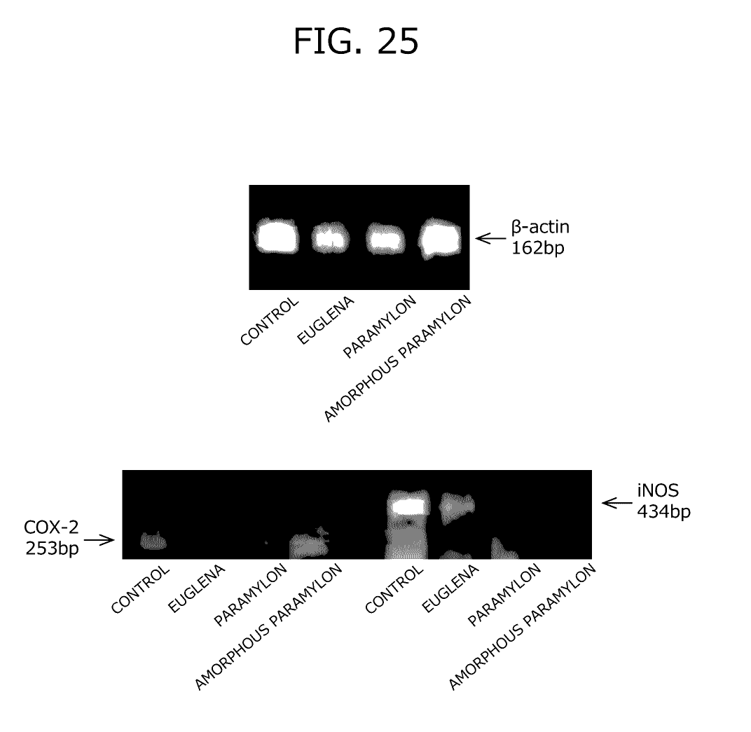

The analysis results are illustrated in FIGS. 25-27. As illustrated in FIG. 25, 434 bp, 253 bp, and 162 bp bands were seen, and iNOS mRNA, COX-2 mRNA, and β-actin mRNA were detected as PCR products. iNOS and COX-2 were normalized to β-actin. The figures illustrate relative indexes of the respective groups with the value of the control considered as 1.0.

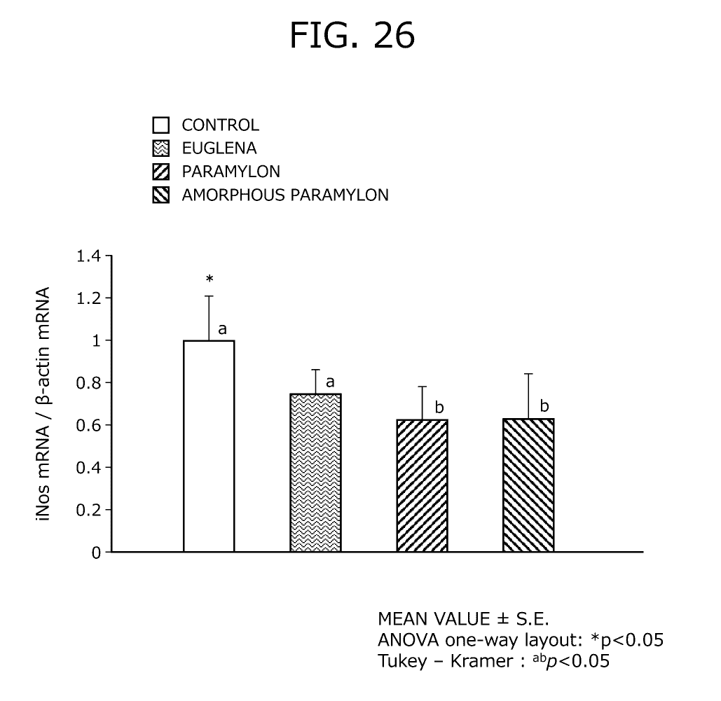

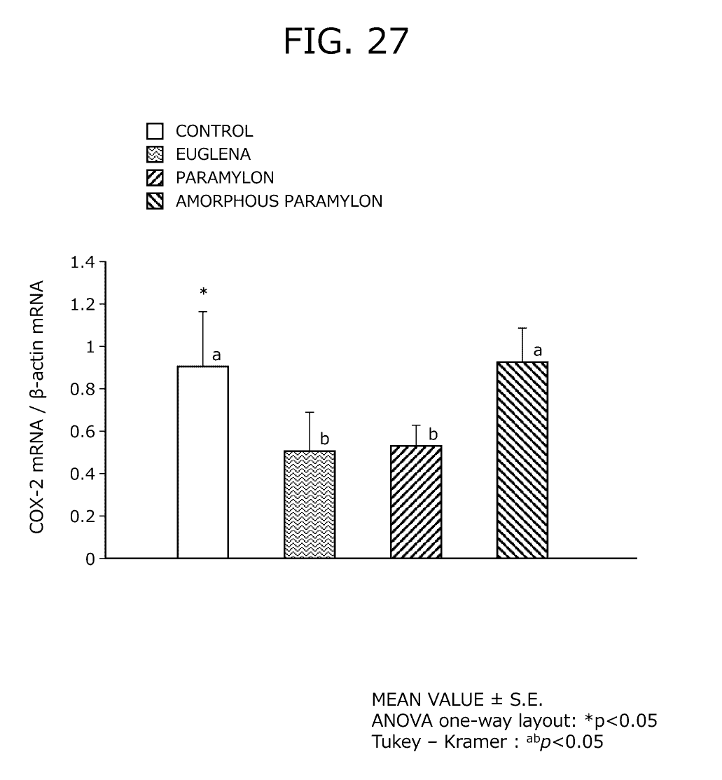

FIG. 26 illustrates iNOS/β-actin, while FIG. 27 illustrates COX-2/β-actin. The results in FIG. 26 indicate that the Euglena group, the paramylon group, and the amorphous paramylon group exhibited inhibited expression of iNOS, compared with the control group. Particularly, the paramylon group and the amorphous paramylon group exhibited a significant inhibition (p<0.05 by Turkey-Kramer test).

The COX-2/β-actin in FIG. 25 is illustrated in FIG. 27. As illustrated in FIG. 27, the Euglena group and the paramylon group exhibited significantly inhibited expression of COX-2 compared with the control group (p<0.05 by Turkey-Kramer test).

Administration of Euglena, paramylon, or amorphous paramylon has been found to inhibit expression of iNOS and COX-2, and thus it is expected that the administration reduced oxidative damage due to stress, thereby preventing a gastric ulcer.

Thus, it has been found that Euglena, paramylon, and amorphous paramylon provide anti-inflammatory effect through inhibition of expression of iNOS, which leads to excessive inflammation, and/or inhibition of expression of COX-2, which is a rate-limiting enzyme in biosynthesis of PG, which leads to excessive inflammation.

The Euglena, the paramylon, and the amorphous paramylon in this Example have been shown to have the effect of inhibiting expression of iNOS and/or expression of COX-2. Thus, it has been found that the Euglena, the paramylon, and the amorphous paramylon in this Example can be used as an iNOS expression inhibitor, a COX-2 expression inhibitor, and an anti-inflammatory agent.

### Study Example 7

### Study of Effect on Rheumatoid Arthritis in Mouse Models of Collagen Arthritis

The effect of the test materials in Examples 1-4 on rheumatoid arthritis was studied in mouse models of collagen arthritis.

Mice (DBA/1J Jms Slc (SPF), 6 week old male, Japan SLC, Inc.) were used as the test animals.

Chicken type II collagen (SIGMA) was dissolved in 0.01 M aqueous acetic acid to a concentration of 2 mg/mL. Then, an equal volume of Freund's complete adjuvant (Difco) was added to the resultant solution to prepare an emulsion (1 mg/mL of collagen), which was intradermally administered into the base of the tail of the mice in an amount of 0.1 mL (0.1 mg of collagen) under isoflurane inhalation anesthesia to sensitize the mice to the collagen. After 3 weeks, the same administration was carried out to boost the mice. And untreated animals were not sensitized and boosted.

The mice were classified into an untreated group, a control group, a Euglena group, a paramylon group, an amorphous paramylon group, and an emulsion paramylon group (n=5 in each of the groups). The test materials in Examples 1-4 were admixed with CE-2 solid diet (CLEA Japan, Inc.) at a concentration of 2% and were given to the mice of the Euglena group, the paramylon group, the amorphous paramylon group, and the emulsion paramylon group orally ad libitum every day from day 5 after the boosting.

From the day of sensitization to the collagen (hereinafter referred to as “sensitization day”), arthritis symptoms in the four limbs were scored by visual inspection, and the total scores for the four limbs were calculated.

The scoring was conducted three times per week (Mondays, Wednesdays, and Fridays) according to arthritis scoring criteria in Table 6 with the reference to scoring by Kakimoto et al. (Shinsei Kagaku Jikken Koza 12, Bunshi Mennekigaku II, Tokyo Kagaku Dojin, 360-372, 1989), and the total scores for the four limbs were calculated.

TABLE 6
Arthritis Scores

| Score | Symptoms |
|---|---|
| 0 | No symptoms |
| 1 | Mild redness and swelling of a small joint of a finger |
| | or the like |
| 2 | Redness and swelling of 2 or more small joints or a |
| | large joint |
| 3 | Redness and swelling of a limb |
| 4 | Intense redness and swelling of an overall limb |

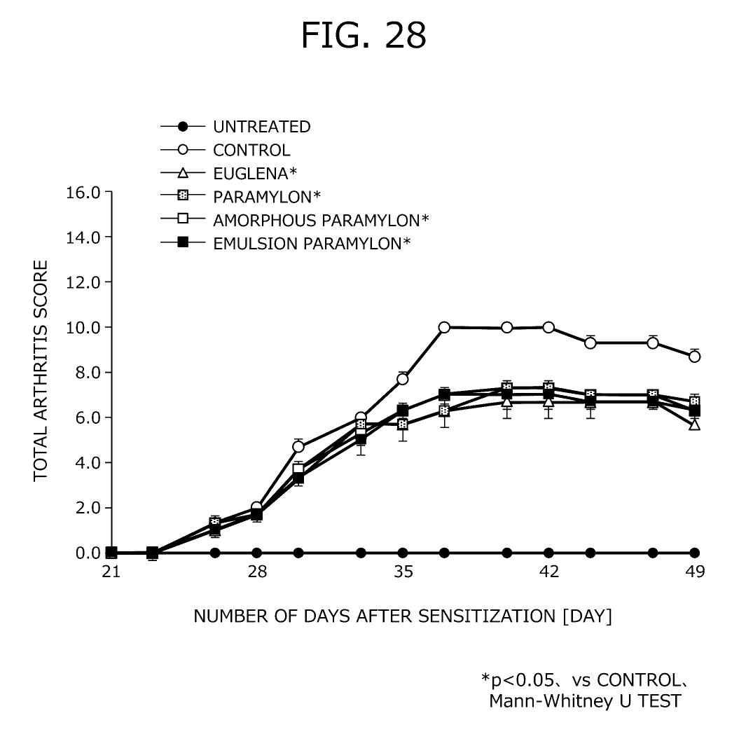

The results of the arthritis scores are illustrated in FIG. 28.

At the final scoring day, all of the Euglena group, the paramylon group, the amorphous paramylon group, and the emulsion paramylon group exhibited a significant lower score compared with the control group, which confirms that continuous administration of the test materials alleviates the symptoms of arthritis.

After 7 weeks from the sensitization day, the animals were subjected to a laparotomy under isoflurane inhalation anesthesia to collect the blood from the abdominal vena cava. The resultant blood was centrifuged to separate the serum, and collagen IgG in the serum was quantified (by ELISA).

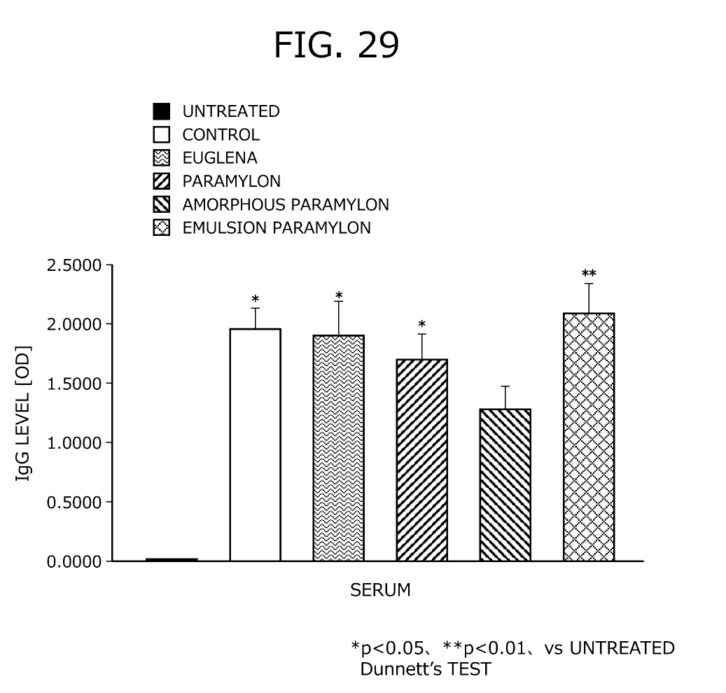

The measurements of the anti-collagen IgG are illustrated in FIG. 29.

While the emulsion paramylon group exhibited a level similar to the level in the control group, while the Euglena group, the paramylon group, and the amorphous paramylon group exhibited a lower level compared with the control group.

The animals that had been subjected to blood collection after 7 weeks from the sensitization day were sacrificed by exsanguinations after the collection. The inguinal lymph nodes (in all of the groups except for the untreated group) and then knee joints (both sides in all of the groups) were removed.

With regard to the inguinal lymph nodes, the separated lymphocytes were divided into three equal portions and individually cultured in medium supplemented with anti-CD3 antibodies. After about 48 hours from the initiation of the cultivation, the culture supernatant was collected, and the levels of cytokines (IL-17A and IFN-γ) secreted in the culture supernatant were measured by multiplex suspension array.

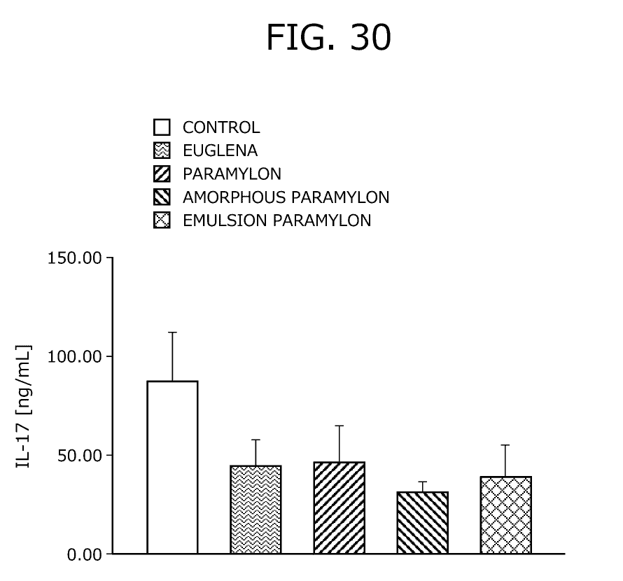

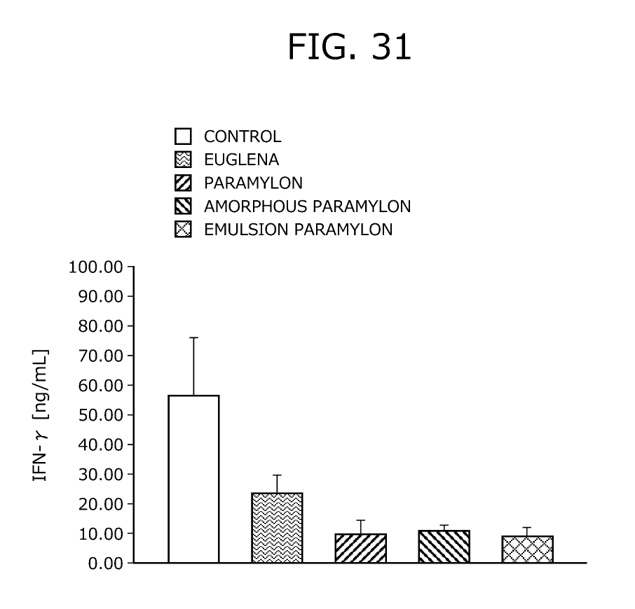

The results are illustrated in FIG. 30 and FIG. 31.

All of the Euglena group, the paramylon group, the amorphous paramylon group, and the emulsion paramylon group exhibited lower levels of IL-17A and IFN-γ cytokines compared with the control group.

The results in FIG. 30 and FIG. 31 have confirmed that the Euglena group, the paramylon group, the amorphous paramylon group, and the emulsion paramylon group reduced IL-17A and IFN-γ, which are cytokines produced by Th1/Th17 and that the immune balance between Th1, Th2, and Th17 was regulated so that immune responses induced by Th1 or Th17 were inhibited, i.e., so that immune responses induced by Th2 become relatively dominant over immune responses induced by Th1 or Th17.

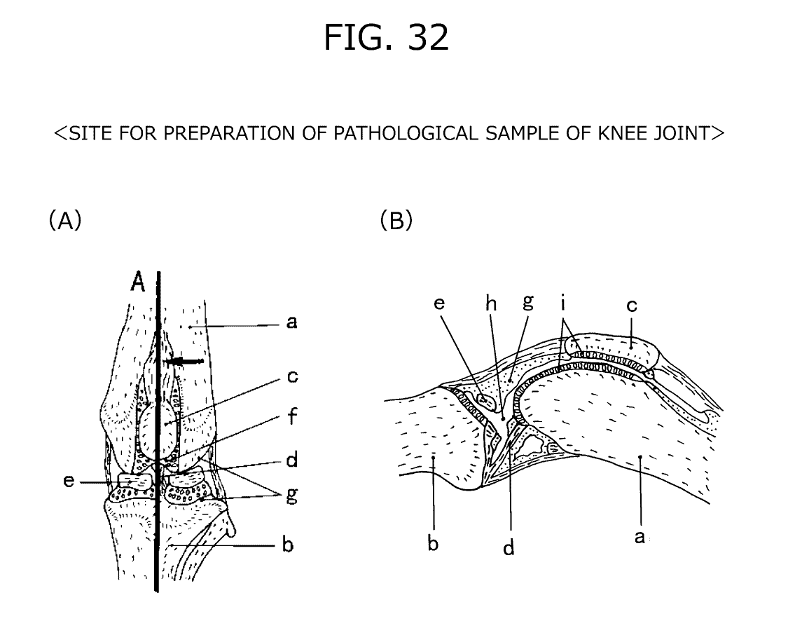

The removed left-knee joints were fixed in 10% neutral buffered formalin. After decalcification in a 10% formic acid-formalin solution, the joints were cut along the line A in the trochlear groove of the femur as illustrated in FIG. 32 (A). Paraffin sections were cut and stained with HE.

The knee joint tissue was rated according to the following criteria: the synovial membrane tissue was rated for edema, inflammatory cell infiltration, synoviocyte proliferation, granulation tissue formation, fibrosis, and exudate in the joint cavity, and the tissue in the trochlear groove of the femur was rated for pannus formation, destruction of the joint cartilage (degeneration and fibrosis), bone destruction (absorption), and osteophyte formation (reactive osteoid formation) on a scale of from 0 to 4 with 0 representing no change, 1 representing very mild, 2 representing mild, 3 representing moderate, and 4 representing severe.

The animals that had a similar average total score for the four limbs were selected as the representatives of each of the groups. Two representatives were selected from the untreated group and the control group, while three representatives were selected from the test material groups.

The results are illustrated in Table 7 and Table 8.

TABLE 7

| | | | |
|---|---|---|---|
| Organ/ | Founding | Gender | Male |

| Tissue | Group | Untreated | | | | Control | | | | Euglena | | | |
|---|---|---|---|---|---|---|---|---|---|---|---|---|---|
| | Dose (%, | 0 | | | | 0 | | | | 2 | | | |
| | Diet | | | | | | | | | | | | |
| | Admixture) | | | | | | | | | | | | |
| | Number of | 2 | | | | 2 | | | | 3 | | | |
| | Animals | | | | | | | | | | | | |
| | Extent | 0 | 1 | 2 | 3 | 0 | 1 | 2 | 3 | 0 | 1 | 2 | 3 |
| | (Rating) | | | | | | | | | | | | |

| | | | | | | | | |
|---|---|---|---|---|---|---|---|---|
| Synovial | Edema | 2 | 1 | 1 | | | 3 | |
| Membrane | Inflammatory Cell Infiltration | 2 | | 1 | | 1 | 3 | |
| | Synoviocyte Proliferation | 2 | 1 | | 1 | | 3 | |
| | Granulation Tissue Formation | 2 | | 2 | | | 2 | 1 |
| | Fibrosis | 2 | 1 | | 1 | | 3 | |
| | Exudate in Joint Cavity | 2 | 1 | | | 1 | 3 | |
| Trochlear | Pannus Formation | 2 | 1 | | 1 | | 3 | |
| Groove of | Destruction of Joint Cartilage | 2 | 1 | | 1 | | 3 | |
| Femur | (Degeneration and Fibrosis) | | | | | | | |
| | Bone Destruction (Absorption) | 2 | 2 | | | | 3 | |
| | Osteophyte Formation (Reactive | 2 | 2 | | | | 3 | |
| | Osteoid Formation) | | | | | | | |

Histopathological Evaluation (Rating)
0: No Significant Change,
1: Very Mild,
2: Mild,
3: Moderate

TABLE 8

| | | | |
|---|---|---|---|
| Organ/ | Founding | Gender | Male |

| Tissue | Group | Paramylon | | | | Amorphous | | | | Emulsion | | | |
|---|---|---|---|---|---|---|---|---|---|---|---|---|---|
| | | | | | | Paramylon | | | | Paramylon | | | |
| | Dose (%, | 2 | | | | 2 | | | | 2 | | | |
| | Diet | | | | | | | | | | | | |
| | Admixture) | | | | | | | | | | | | |
| | Number of | 3 | | | | 3 | | | | 3 | | | |
| | Animals | | | | | | | | | | | | |
| | Extent | 0 | 1 | 2 | 3 | 0 | 1 | 2 | 3 | 0 | 1 | 2 | 3 |
| | (Rating) | | | | | | | | | | | | |

| | | | | | | | | | | |
|---|---|---|---|---|---|---|---|---|---|---|
| Synovial | Edema | 3 | | | 3 | | 2 | 1 | | |
| Membrane | Inflammatory Cell Infiltration | 2 | 1 | | 3 | | | 2 | 1 | |
| | Synoviocyte Proliferation | 1 | 2 | | 3 | | 1 | 1 | | 1 |
| | Granulation Tissue Formation | 2 | 1 | | 2 | 1 | | 2 | 1 | |
| | Fibrosis | 1 | 1 | 1 | 3 | | | 3 | | |
| | Exudate in Joint Cavity | 3 | | | 3 | | 3 | | | |
| Trochlear | Pannus Formation | 2 | 1 | | 3 | | 2 | 1 | | |
| Groove of | Destruction of Joint Cartilage | 1 | 1 | 1 | 3 | | 2 | 1 | | |
| Femur | (Degeneration and Fibrosis) | | | | | | | | | |
| | Bone Destruction (Absorption) | 3 | | | 3 | | 3 | | | |
| | Osteophyte Formation (Reactive | 3 | | | 3 | | 3 | | | |
| | Osteoid Formation) | | | | | | | | | |

Histopathological Evaluation (Rating)
0: No Significant Change,
1: Very Mild,
2: Mild,
3: Moderate

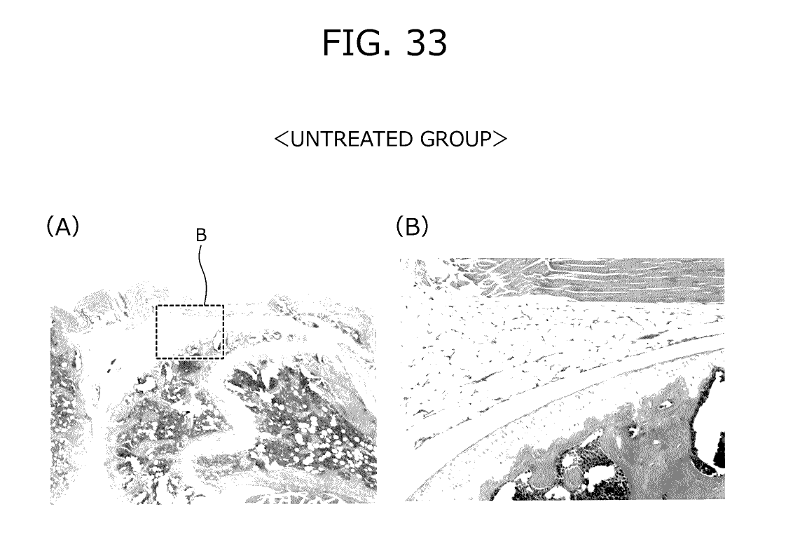

The photographs of pathological samples of the tissue of the left knee joints of the representatives of each of the groups are illustrated in FIGS. 33-38.

In the untreated group, as illustrated in FIG. 33, there were no findings that might be attributed to arthritis in the synovial membranes and the trochlear grooves of the femurs.

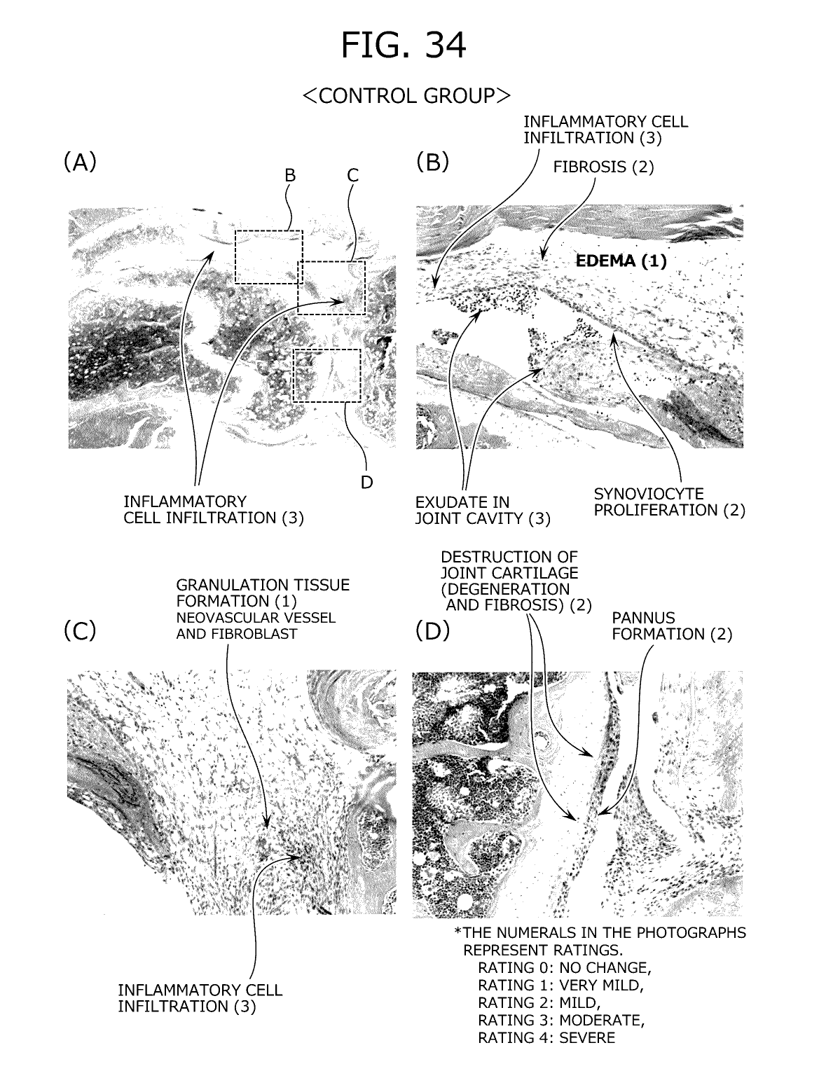

In the control group illustrated in FIG. 34, the synovial membranes had edema, inflammation, granulation tissue formation, fibrosis, and exudates rated as very mild (rating 1) to moderate (rating 2). And the trochlear grooves of the femurs had pannus formation and cartilage destruction rated as mild (rating 2).

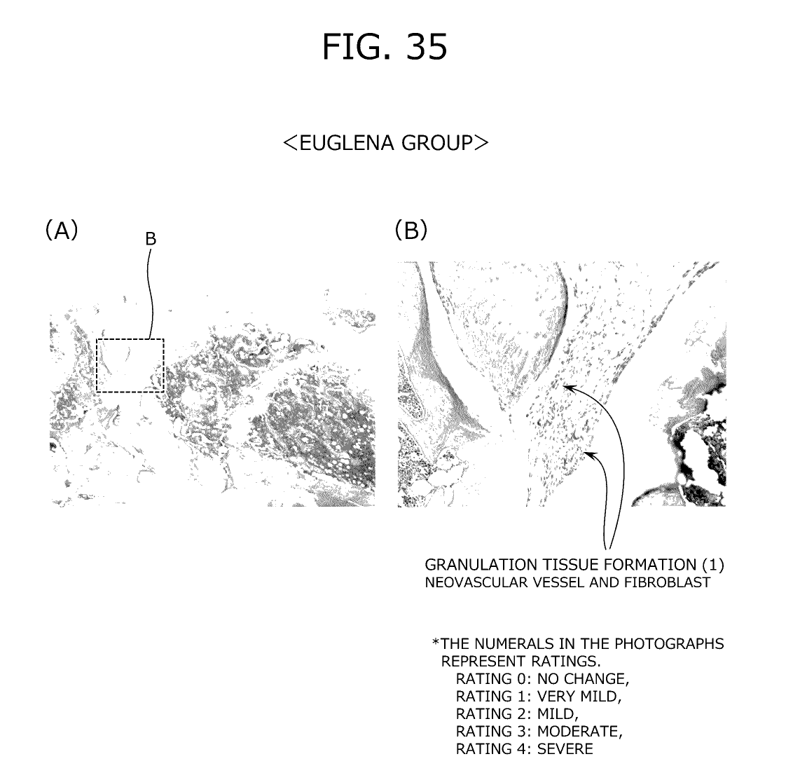

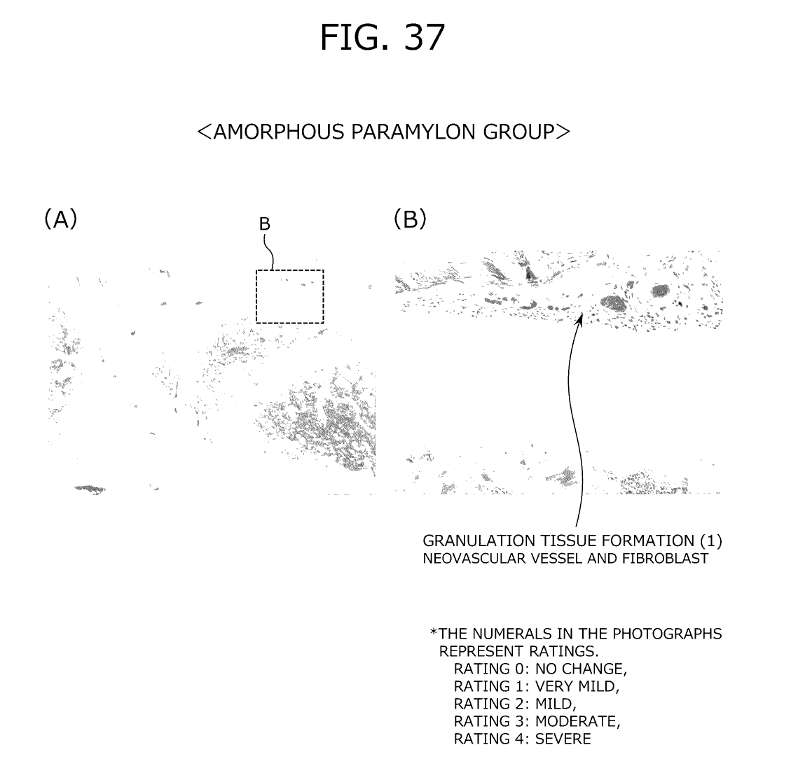

In the Euglena group illustrated in FIG. 35 and the amorphous paramylon group illustrated in FIG. 37, no findings were observed except that the synovial membranes had granulation tissue formation rated as very mild (rating 1).

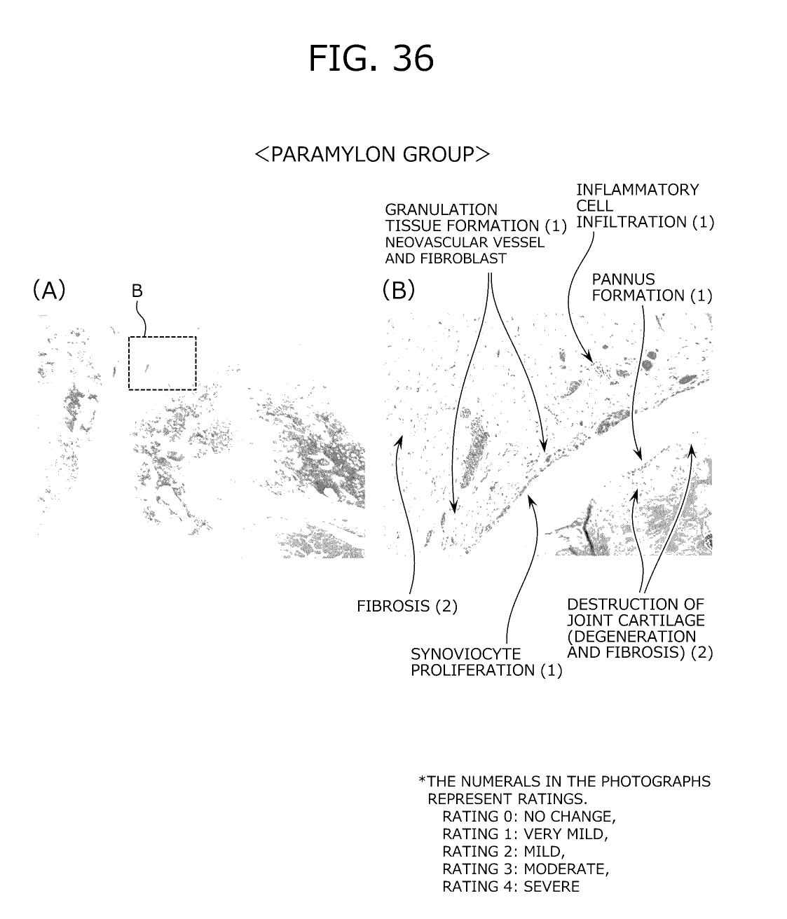

In the paramylon group illustrated in FIG. 36, the synovial membranes had inflammation, granulation tissue formation, and fibrosis rated as very mild (rating 1) or mild (rating 2), and the trochlear grooves of the femurs had pannus formation and the cartilage destruction rated as very mild (rating 1) or mild (rating 2).

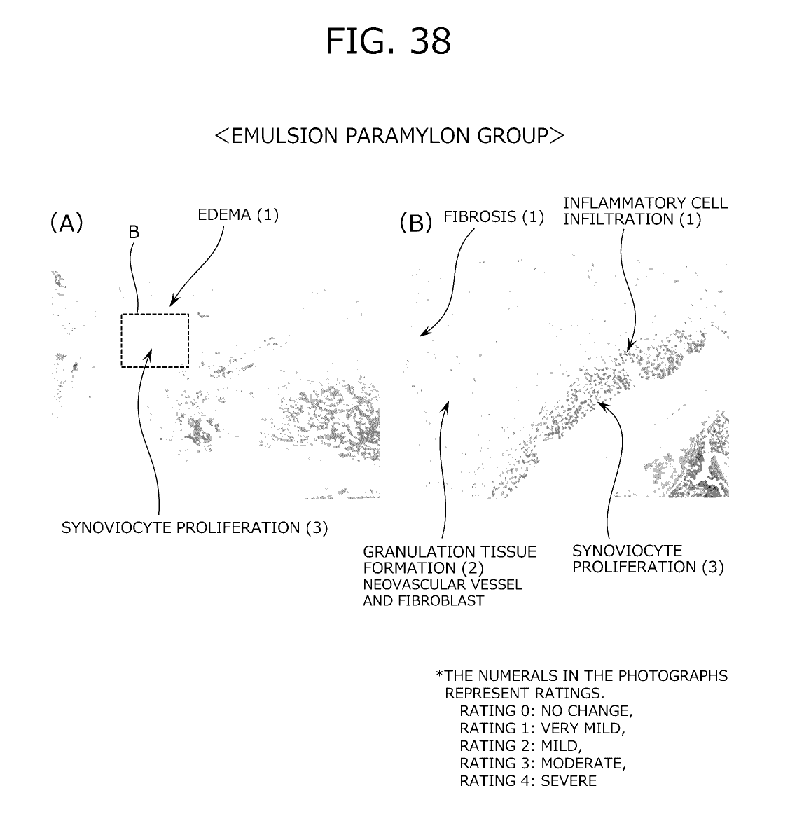

In the emulsion paramylon group illustrated in FIG. 38, the synovial membranes had edema, inflammation, granulation tissue formation, fibrosis, and exudates rated as very mild (rating 1), as similar to the control group. The trochlear grooves of the femurs had pannus formation and cartilage destruction rated as mild (rating 2).

The inguinal lymph nodes removed after 7 weeks from the sensitization day were analyzed on a flow cytometer for distribution of the cell population. The suppressor T cells were measured using Mouse Th17/Treg Phenotyping Kit (BD pharmingen).

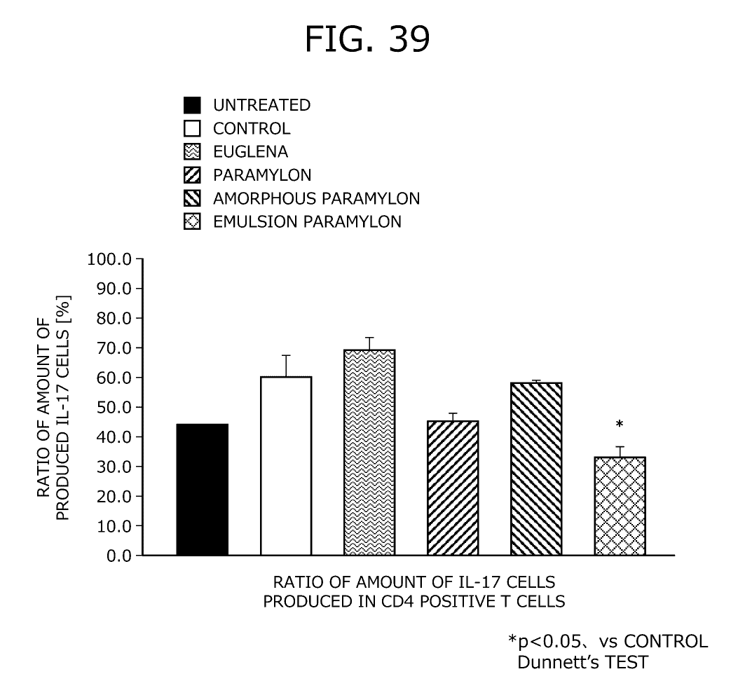

FIG. 39 illustrates the ratio of IL-17 production in CD4 positive T cells, as determined by analysis on a flow cytometer.

FIG. 39 indicates that the paramylon group and the amorphous paramylon group exhibited a smaller number of IL-17A produced, compared with the control group. The emulsion paramylon group exhibited a significantly smaller number, compared with the control group.

In the control group, the arthritis scores increased over time, and the levels of IgG in the serum increased. In the histopathological study of the knee joints, the synovial membranes had inflammation, granulation tissue formation, fibrosis, and exudates rates as very mild to moderate, and the trochlear grooves of the femurs had pannus formation and cartilage destruction rated as mild.

With regard to the above conditions, the Euglena group, the paramylon group, the amorphous paramylon group, and the emulsion paramylon group tended to exhibit low visual scores compared with the control group, and the statistically significant differences were also observed. With regard to the levels of IgG in the serum, the amorphous paramylon group exhibited lower levels.

In the histopathological study, no findings were observed in the Euglena group and the amorphous paramylon group except that the synovial membranes had granulation tissue formation rated as very mild. In the paramylon group, although the synovial membranes had inflammation, granulation tissue formation, and fibrosis, and the trochlear grooves of the femurs had pannus formation and cartilage destruction, their extent was less severe, compared with the control group.

All of the Euglena group, the paramylon group, the amorphous paramylon group, and the emulsion paramylon group exhibited decreased secretion of the cytokines in the lymphoid culture supernatant by stimulation with Anti-CD3, compared with the control group. Particularly, the Euglena group and the amorphous paramylon group showed a remarkable reduction.

As described above, the study of the effects of the Euglena-derived materials on autoimmune diseases in mouse models of collagen arthritis has revealed that the Euglena group, the paramylon group, the amorphous paramylon group, and the emulsion paramylon group had the effect of preventing the onset of arthritis. Particularly, the Euglena group and the amorphous paramylon group had a greater prevention effect, which was histologically distinct.

### REFERENCE SIGNS LIST

a femur
b tibia
c patella
d posterior cruciate ligament
e meniscus
f anterior cruciate ligament

1. A method for improving a physical constitution that has an immune imbalance of Th1, Th2, and Th17 shifted towards a Th1, a Th2 and/or a Th17 dominance, the method comprising administering to a subject in need of immune balance an effective amount an immune balance regulator comprising:
a Euglena-derived material,
wherein the immune balance regulator regulates an immune balance between Th1, Th2, and Th17, the immune balance being a balance between immune responses individually induced by Th1, Th2, and Th17 in a living body, and
wherein the improving a physical constitution is preventing or treating influenza, peptic ulcer, or articular rheumatism.

2. The method according to claim 1, wherein the regulator regulates the immune balance between Th1, Th2, and Th17 so that immune responses induced by Th1 become relatively dominant over immune responses induced by Th2 or Th17.

3. The method according to claim 1, wherein the immune imbalance of Th1, Th2, and Th17 is shifted towards a Th2 dominance.

4. The method according to claim 1, wherein the physical constitution that has the immune imbalance of Th1, Th2, and Th17 shifted towards the Th2 dominance is a physical constitution that is susceptible to an infectious or a stress disease.

5. (canceled)

6. The method according to claim 4, wherein the regulator is administered prior to expected onset of the infectious or stress disease.

7. (canceled)

8. The method according to claim 4, wherein the disease is influenza or peptic ulcer.

9. (canceled)

10. The method according to claim 1, wherein the regulator increases the ratio IFN-γ production to IL-4 production in the living body.

11. The method according to claim 1, wherein the regulator promotes the production of IFN-γ and inhibits the production of IL-4, IL-5, and IL-10 in the living body.

12. The method according to claim 1, wherein the regulator regulates the immune balance of Th1, Th2, and Th17 so that immune responses induced by Th2 become relatively dominant over immune responses induced by Th1 or Th17.

13. The method according to claim 1, wherein
the immune imbalance of Th1, Th2, and Th17 is shifted towards a Th1 and/or a Th17 dominance and the improving the physical constitution comprises preventing or treating rheumatoid arthritis.

14. The method according to claim 1, wherein the Euglena-derived material is paramylon or a processed product therefrom.

15. A food composition comprising:
a Euglena-derived material,
wherein the food composition regulates the immune balance between Th1, Th2, and Th17, the immune balance being a balance between immune responses individually induced by Th1, Th2, and Th17 in a living body.
